(19) United States
(12) Patent Application Publication
(10) Pub. No.: US 2005/0287103 A1
(43) Pub. Date: Dec. 29, 2005

(54) Cosmetic composition comprising at least one ester and at least one film-forming polymer

(76) Inventors: Vanina Filippi, Paris (FR); Caroline Lebre, Thiais (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,269

(22) Filed: Jun. 8, 2005

Related U.S. Application Data
(60) Provisional application No. 60/580,103, filed on Jun. 17, 2004.

(30) Foreign Application Priority Data
Jun. 8, 2004 (FR) 04 06175

(51) Int. Cl. A61K 7/075

(52) U.S. Cl. 424/70.22

(57) ABSTRACT

Disclosed herein is a cosmetic composition for making up and/or caring for the skin, the lips and/or the integuments, comprising, in a physiologically acceptable medium, at least one ester of a diol dimer and of a diacid dimer of unsaturated fatty acids and of diol dimer, and at least one film-forming polymer.

This application claims benefit of U.S. Provisional Application No. 60/580,103, filed Jun. 17, 2004, the contents of which are incorporated herein by reference. This application also claims benefit of priority under 35 U.S.C. § 119 to French Patent Application No. 04 06175, filed Jun. 8, 2004, the contents of which are also incorporated by reference.

Disclosed herein is a cosmetic composition, such as a cosmetic composition for making up and/or caring for the skin, including the scalp, of the human face and/or body, and human lips and/or integuments, for instance the hair, the eyelashes, the eyebrows, and the nails, comprising a cosmetically acceptable medium.

The composition disclosed herein may be a makeup product for the human body, lips, and/or integuments having, for example, non-therapeutic care and/or treatment properties. The composition may be chosen from lipsticks, lip glosses, makeup rouges, eyeshadows, tattoo products, mascaras, eyeliners, nail varnishes, artificial skin tanning products, hair-coloring products, and haircare products.

There are many cosmetic compositions for which the gloss properties of the deposited film, after application to the keratin materials (such as skin, lips, and integuments), may be desirable. Examples that may be mentioned include lipsticks, nail varnishes, and certain hair products. To achieve such gloss properties, the formulator may use, as active principle in terms of gloss, lanolins combined with at least one “glossy” oil, for instance a) oily polymers such as polybutenes that have a high viscosity, b) esters of fatty acid or of fatty alcohol with a high carbon number (typically greater than 16), and c) certain plant oils.

However, the glossy compositions disclosed in the art may have the drawback of having insufficient staying power over time. These compositions, when applied to the skin and/or the lips, may be impaired during contact with liquids, such as saliva, sebum, water, drinks, and oils, such as the edible oils consumed, for example, during a meal.

It would thus be desirable to have cosmetic makeup and/or care compositions that form a deposit that has good staying power on contact with liquids brought into contact with the makeup and/or care composition, for example during a meal.

The present inventors have found that, in at least one embodiment disclosed herein is a combination comprising at least one ester of a diol dimer with a specific dicarboxylic diacid, may give satisfactory gloss without, however, affecting the staying power of the cosmetic composition.

Esters of diol dimers and of monocarboxylic or dicarboxylic acids have been described in general in French Patent No. FR 2 795 309 as being useful for preparing cosmetic compositions having improved stability properties. More recently, documents JP 2002-128 623, JP 2002-128 628, and JP 2002-128 629 proposed cosmetic compositions, such as makeup compositions, including as gloss active agent, esters of dilinoleic diacids with dilinoleyl diol dimers.

The present disclosure, and at least certain embodiments disclosed herein, are based on the observation by the inventors that a composition comprising a combination of at least one ester of a diol dimer and of acid with at least one film-forming polymer may be glossy and may have good staying power.

Consequently, the present disclosure relates to a cosmetic composition comprising, in a physiologically acceptable medium, at least one ester of a diol dimer and of at least one C₄ to C₃₄ monocarboxylic or dicarboxylic acid, and at least one film-forming polymer.

The present disclosure also relates to a process for making up and/or caring for the skin, the lips and/or the integuments, comprising applying at least one composition as disclosed herein to the skin, the lips, and/or the integuments.

The diol dimer esters and acid esters that may be used herein may be commercially available or may be prepared in a conventional manner. They may be of plant origin and may be obtained by esterification of a diol dimer with a C₄-C₃₄ monocarboxylic acid, for instance a fatty acid, or with a dicarboxylic acid such as a diacid dimer.

In the case of esterification with a monocarboxylic acid, diol dimer esters and acid esters of relatively high molecular weight, ranging from about 1,000 to 1,300 g/mol, may be obtained. For example, a diol dimer dicarboxylate with a weight-average molecular weight, determined by gel permeation chromatography (GPC), ranging from 2,000 to 20,000 g/mol, such as from 2,000 to 4,000 g/mol, may be obtained.

The monocarboxylic acid that may be used according to the present disclosure contains from 4 to 34 carbon atoms, such as from 10 to 32 carbon atoms.

By way of illustration of monocarboxylic acids that are suitable for use herein, mention may be made of:
saturated linear acids such as butanoic acid, pentanoic acid, hexanoic acid, heptanoic acid, octanoic acid, nonanoic acid, decanoic acid, undecanoic acid, dodecanoic acid, tridecanoic acid, tetradecanoic acid, heptadecanoic acid, hexadecanoic acid, pentadecanoic acid, octadecanoic acid, nonadecanoic acid, eicosanoic acid, docosanoic acid, and tetracosanoic acid;
branched fatty acids, for instance isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyidodecanoic acid, 2-decyltetradecanoic acid, 2-dodecylhexadecanoic acid, 2-tetradecyloctadecanoic acid, 2-hexadecyloctadecanoic acid, and long-chain fatty acids obtained from lanolin;
unsaturated linear C₈ to C₃₄ fatty acids, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, and arachidonic acid;
hydroxy acids such as 2-hydroxybutanoic acid, 2-hydropentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxytetradecanoic acid, 2-hydroxyhexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid, and 2-hydroxytetracosanoic acid;
cyclic acids such as cyclohexanoic acid, hydrogenated rosin, rosin, abietic acid, hydrogenated abietic acid, benzoic acid, p-oxybenzoic acid, p-aminobenzoic acid, cinnamic acid, p-methoxycinnamic acid, salicylic acid, gallic acid, pyrrolidonecarboxylic acid, and nicotinic acid; and
fatty acids of natural origin, such as: the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil, and of mink oil, and mixtures thereof.

In one embodiment, the monocarboxylic acid may be a fatty acid, as defined above. As used herein, the term “fatty acid” means a carboxylic acid obtained by hydrolysis of plant oils or animal fats or oils. The fatty acid may be saturated or unsaturated.

The ester obtained may be at least one of a diester and a monoester. In the present case, the ester may be a mixture of at least two types of esters formed with different carboxylic acids.

The dicarboxylic acid that may be used according to certain embodiments may contain at least two carboxylic groups per molecule. It may be represented by formula (I) below:
HOOC—(CH₂)ₙ—COOH (I)
in which n is an integer ranging from 1 to 16, such as from 3 to 16.

As non-limiting illustrations of dicarboxylic acids that are suitable for certain embodiments disclosed herein, mention may be made of malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradecamethylenedicarboxylic acid, 1,15-pentadecamethylenedicarboxylic acid, and 1,16-hexadecamethylenedicarboxylic acid, and mixtures thereof.

The dicarboxylic acid may also be a diacid dimer. As used herein, the term “diacid dimer” denotes a diacid obtained by polymerization reaction, such as by intermolecular dimerization of at least one unsaturated monocarboxylic acid.

The dicarboxylic acid may be derived from the dimerization of an unsaturated fatty acid, such as a C₈ to C₃₄, C₁₂ to C₂₂, C₁₆ to C₂₀, or C₁₈ fatty acid.

By way of representation of these unsaturated fatty acids, mention may be made, as stated above, of undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid, and mixtures thereof.

According to one embodiment, mention may be made of the diacid dimer from which the diol dimer to be esterified is also derived.

For example, a diacid dimer may be obtained by dimerization of linoleic acid, optionally followed by hydrogenation of the carbon-carbon bonds. The diacid dimer may be in saturated form, i.e., it may comprise no carbon-carbon double bonds. According to another embodiment, the possible carbon-carbon double bonds of the diacid dimer are totally or partially hydrogenated, after esterification reaction of the diacid dimer with the diol dimer.

According to one embodiment, the diacid dimer is a commercial product comprising a dicarboxylic acid containing about 36 carbon atoms. This product may also comprise a trimeric acid and a monomeric acid, in proportions that depend on the degree of purity of the product. Conventionally, products with a diacid dimer content of greater than 70% and others whose diacid dimer content has been adjusted to 90% or more are commercially available.

Diacid dimers such as dilinoleic diacids whose stability towards oxidation has been improved by hydrogenation of the double bonds remaining after the dimerization reaction are also commercially available.

Any diacid dimer that is currently commercially available may be used herein.

In an esterification reaction with a dicarboxylic acid such as a diacid dimer, the mean degree of esterification and the average molecular weight of the ester obtained may be adjusted by varying the ratio of the diol dimer to the dicarboxylic acid, such as the diacid dimer.

The ratio, expressed as the molar proportion of a dicarboxylic acid based on the average molecular weight calculated from its acid number per 1 mol of diol dimer based on the average molecular weight calculated from its hydroxyl number, may range from 0.2 to 1.2 mol, such as from 0.4 to 1.0 mol, for example equal to 0.5 or 0.7 mol.

As used herein, the term “diol dimer” may denote saturated diols produced by hydrogenation of the corresponding diacid dimers, a diacid dimer being as defined above.

As regards the diol dimer manufactured industrially, it may also comprise other components, for example a triol trimer, a monoalcohol, and compounds of ether type, depending on the degree of purification of the dimeric acid and/or of the lower alcohol ester thereof, used as starting material. Generally, products whose diol dimer content is greater than 70% may be used in accordance with certain embodiments disclosed herein. However, a diol dimer of high purity may be used, such as a compound whose diol dimer content is greater than 90%.

Thus, a diol dimer may be produced by catalytic hydrogenation of a diacid dimer, which is itself obtained by dimerization of at least one unsaturated fatty acid, for example of C₈ to C₃₄, such as those mentioned above, of C₁₂ to C₂₂, of C₁₆ to C₂₀, or of C₁₈, such as, for example, oleic acid and linoleic acid.

According to one embodiment, the diol dimer is derived from the hydrogenation of the acid functions of dilinoleic diacid.

In certain embodiments, the diol dimer may be obtained by dimerization of linoleic acid, followed by hydrogenation of the acid functions. The diol dimer may be in saturated form, i.e., it may comprise no carbon-carbon double bonds. According to another embodiment, the possible carbon-carbon double bonds of the diol dimer are totally or partially hydrogenated, after esterification reaction of the diacid dimer with the diol dimer.

According to one embodiment disclosed herein, the diol dimer ester is an ester of a diol dimer and of a diacid dimer and is, for example, a compound of general formula (II)
HO—R¹—(—OCO—R²—COO—R¹)ₕ—OH (II)
in which:
R¹ is a diol dimer residue obtained by hydrogenation of dilinoleic diacid,

R² is a hydrogenated dilinoleic diacid residue, and
h is an integer ranging from 1 to 9.

By way of illustration of esters that are suitable for embodiments disclosed herein, mention may be made of the esters of dilinoleic diacids and of dilinoleic diol dimers sold by the company Nippon Fine Chemical under the trade name Lusplan DD-DA5 and DD-DA7.

The amount of ester may be adjusted so as to control the mean gloss of the composition to the desired value. In the present case, the ester may be present in an amount ranging from 1% to 99% by weight, such as from 2% to 60% by weight, from 5% to 40%, or from 10% to 35% by weight, relative to the total weight of the composition.

Film-Forming Polymer

As used herein, the term “film-forming” polymer means a polymer capable, by itself or in the presence of an auxiliary film-forming agent, of forming a continuous film that adheres to a support, such as keratin materials, and may be a cohesive film, such as a film whose cohesion and mechanical properties are such that the film may be isolated from the support.

Among the at least one film-forming polymer that may be used in the composition disclosed herein, mention may be made of synthetic free-radical polymers, synthetic polycondensate polymers, and polymers of natural origin. Film-forming polymers that may be mentioned include acrylic polymers, polyurethanes, polyesters, polyamides, polyureas, and cellulose-based polymers, for instance nitrocellulose.

In one embodiment, the organic film-forming polymer is at least one polymer chosen from:
film-forming polymers that are soluble in the liquid fatty phase, such as liposoluble polymers, when the liquid fatty phase comprises at least one oil;
film-forming polymers that are dispersible in the liquid fatty phase, different from grafted ethylenic polymers, such as polymers in the form of non-aqueous dispersions of polymer particles, for example dispersions in silicone oils or hydrocarbon-based oils. In one embodiment, the non-aqueous polymer dispersions comprise surface-stabilized polymer particles;
aqueous dispersions of film-forming polymer particles, often known as “latices”. In this case, the composition should comprise, besides the liquid fatty phase, an aqueous phase;
water-soluble film-forming polymers. In this case, the composition should comprise, besides the liquid fatty phase, an aqueous phase.

The composition disclosed herein may comprise, as the at least one film-forming polymer, a dispersion of particles of a grafted ethylenic polymer in a liquid fatty phase.

As used herein, the term “ethylenic” polymer means a polymer obtained by polymerization of ethylenically unsaturated monomers.

The dispersion of grafted ethylenic polymer may be free of stabilizing polymer different from the grafted polymer, such as those described in European Patent No. EP 749 747 and described hereinbelow, and the particles of grafted ethylenic polymer therefore may not be surface-stabilized with such additional stabilizing polymers. The grafted polymer may therefore be dispersed in the liquid fatty phase in the absence of additional surface stabilizer for the particles.

As used herein, the term “grafted” polymer means a polymer having a skeleton comprising at least one side chain that is pendent or located at the end of a chain, for example pendent.

In certain embodiments, the grafted ethylenic polymer comprises an ethylenic skeleton that is insoluble in the liquid fatty phase and side chains covalently bonded to the skeleton, which are soluble in the liquid fatty phase.

The grafted ethylenic polymer may be a non-crosslinked polymer. In certain embodiments, the polymer is obtained by polymerization of monomers comprising one polymerizable group.

According to one embodiment disclosed herein, the grafted ethylenic polymer is a grafted acrylic polymer.

The grafted ethylenic polymer may be obtained by free-radical polymerization in an organic polymerization medium:
of at least one ethylenic monomer, such as an ethylenic monomer of at least one acrylic monomer and optionally of at least one additional non-acrylic vinyl monomer, to form the said insoluble skeleton; and
of at least one macromonomer comprising a polymerizable end group to form the side chains, the said macromonomer having a weight-average molar mass of greater than or equal to 200 and the polymerized macromonomer being present in an amount ranging from 0.05% to 20% by weight relative to the total weight of the polymer.

The liquid fatty phase may contain the organic polymerization medium for the grafted ethylenic polymer.

The organic liquid dispersion medium, corresponding to the medium in which the grafted polymer is supplied, may be identical to the polymerization medium.

However, the polymerization medium may be totally or partially replaced with another organic liquid medium. This other organic liquid medium may be added, after polymerization, to the polymerization medium. The polymerization medium may then be totally or partially evaporated.

The liquid fatty phase may contain liquid organic compounds other than those present in the dispersion medium. These other compounds are chosen such that the grafted polymer remains in dispersed form in the liquid fatty phase.

The organic liquid dispersion medium is present in the liquid fatty phase of the composition disclosed herein due to the introduction into the composition of the dispersion of grafted polymer obtained.

The liquid fatty phase comprises, and in certain embodiments predominantly comprises, at least one liquid organic compound (or oil) as defined below.

In certain embodiments, the liquid fatty phase is a non-aqueous liquid organic phase that is immiscible with water at room temperature (25° C.).

As used herein, the term “liquid organic compound” means a non-aqueous compound that is in liquid form at room temperature (25° C.) and therefore flows under its own weight.

As used herein, the term “silicone compound” means a compound containing at least one silicon atom.

The composition disclosed herein may comprise at least one volatile oil as described below.

As used herein, the term “volatile oil” means an oil capable of evaporating from the skin, the lips and/or keratin fibers in less than one hour, and having, for example, a vapor pressure, at room temperature and atmospheric pressure, ranging from 10⁻³ to 300 mmHg (0.13 Pa to 40,000 Pa).

The at least one volatile oil may be silicone-based or non-silicone-based. It may be chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, isododecane, isodecane, and isohexadecane, and mixtures thereof.

The volatile oil may be present in an amount ranging from 1% to 70% by weight, such as ranging from 5% to 50% by weight or ranging from 10% to 35% by weight, relative to the total weight of the composition.

The liquid fatty phase may contain at least one non-volatile oil as described below. The at least one non-volatile oil may be present in an amount ranging from 1% to 80% by weight, such as ranging from 5% to 60% by weight or ranging from 10% to 50% by weight, relative to the total weight of the composition.

Among the liquid organic compounds or oils that may be present in the liquid organic dispersion medium, mention may be made of:
liquid organic compounds, such as silicone-based or non-silicone-based liquid organic compounds, having a global solubility parameter according to the Hansen solubility space of less than or equal to 18 (MPa)^{1/2}, such as less than or equal to 17 (MPa)^{1/2}; and
monoalcohols having a global solubility parameter according to the Hansen solubility space of less than or equal to 20 (MPa)^{1/2}; and
mixtures thereof.

The global solubility parameter 8 according to the Hansen solubility space is defined in the article “Solubility parameter values” by Eric A. Grulke in the book “Polymer Handbook”, 3rd Edition, Chapter VII, p. 519-559, by the relationship:
δ=(δ_{D}²+δ_{P}²+δ_{H}²)^{1/2}
in which
δ_{D} characterizes the London dispersion forces arising from the formation of dipoles induced during molecular impacts,
δ_{P} characterizes the Debye interaction forces between permanent dipoles, and
δ_{H} characterizes the forces of specific interactions (such as hydrogen bonding, acid/base, donor/acceptor, etc.).

The definition of solvents in the solubility space according to Hansen is described in the article by C. M. Hansen: “The three-dimensional solubility parameters”, J. Paint Technol. 39, 105 (1967).

Among the liquid organic compounds, such as silicone-based or non-silicone-based liquid organic compounds, having a global solubility parameter according to the Hansen solubility space of less than or equal to 18 (MPa)^{1/2}, mention may be made of liquid fatty substances, such as at least one oil, which may be chosen from natural or synthetic, carbon-based, hydrocarbon-based, fluoro, and silicone oils, which are optionally branched.

Among the at least one oil, mention may be made of plant oils formed from fatty acid esters and from polyols, for example triglycerides, such as sunflower oil, sesame oil, and rapeseed oil, and esters derived from acids or alcohols containing a long chain (i.e., a chain containing from 6 to 20 carbon atoms), such as the esters of formula RCOOR′in which R represents a higher fatty acid residue containing from 7 to 19 carbon atoms and R′ represents a hydrocarbon-based chain containing from 3 to 20 carbon atoms, such as palmitates, adipates, and benzoates, for example diisopropyl adipate.

Mention may also be made of linear, branched and/or cyclic alkanes which may be volatile, such as liquid paraffin, liquid petroleum jelly, hydrogenated polyisobutylene, isododecane, Isopars®, and volatile isoparaffins. Mention may also be made of esters, ethers, and ketones.

Mention may also be made of silicone oils such as polydimethylsiloxanes and polymethylphenylsiloxanes, optionally substituted with aliphatic and/or aromatic groups, which are optionally fluorinated, or with functional groups such as hydroxyl, thiol and/or amine groups, and volatile silicone oils, which may be, for example, cyclic.

Mention may be made of volatile and/or non-volatile, optionally branched silicone oils.

As used herein, the term “volatile oil” means an oil capable of evaporating from the skin or the lips in less than one hour, and having, for example, a vapor pressure, at room temperature and atmospheric pressure, ranging from 10⁻³ to 300 mmHg (0.13 Pa to 40,000 Pa).

As volatile silicone oils that may be used in accordance with certain embodiments, mention may be made of linear or cyclic silicones containing from 2 to 7 silicon atoms, these silicones optionally comprising alkyl or alkoxy groups containing from 1 to 10 carbon atoms. Mention may be made, for instance, of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, and decamethyltetrasiloxane, and mixtures thereof.

Among the non-volatile silicone oils that may be mentioned are non-volatile polydialkylsiloxanes, such as non-volatile polydimethylsiloxanes (PDMS); polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyl trimethylsiloxy diphenylsiloxanes, diphenyl dimethicones, diphenyl methyldiphenyltrisiloxanes, and polymethylphenylsiloxanes; polysiloxanes modified with fatty acids (such as of C₈-C₂₀), fatty alcohols (such as of C₈-C₂₀) and polyoxyalkylenes (such as polyoxyethylene and/or polyoxypropylene); amino polysiloxanes; polysiloxanes containing hydroxyl groups; fluoro polysiloxanes comprising a fluorinated group that is pendent or at the end of a silicone chain, containing from 1 to 12 carbon atoms, all or some of the hydrogen atoms of which are replaced with fluorine atoms; and mixtures thereof.

As non-silicone-based liquid organic compounds with a global solubility parameter according to the Hansen solubility space of less than or equal to 18 (MPa)^{1/2}, mention may be made of:
linear, branched, or cyclic esters containing at least 6 carbon atoms, such as 6 to 30 carbon atoms;
ethers containing at least 6 carbon atoms, such as 6 to 30 carbon atoms; and
ketones containing at least 6 carbon atoms, such as 6 to 30 carbon atoms.

As used herein, the expression “liquid monoalcohols having a global solubility parameter according to the Hansen solubility space of less than or equal to 20 (MPa)^{1/2}” means aliphatic fatty liquid monoalcohols containing from 6 to 30 carbon atoms, the hydrocarbon-based chain not comprising a substitution group. Monoalcohols according to certain embodiments that may be mentioned include oleyl alcohol, decanol, octyldodecanol, and linoleyl alcohol.

According to one embodiment disclosed herein, the liquid fatty phase may be a non-silicone-based liquid fatty phase.

As used herein, the term “non-silicone-based liquid fatty phase” means a fatty phase comprising at least one non-silicone-based liquid organic compound or oil, such as those mentioned above, the non-silicone compounds being predominantly present in the liquid fatty phase, i.e., present in an amount of at least 50% by weight, such as from 50% to 100% by weight, from 60% to 100% by weight, from 60% to 99% by weight, from 65% to 100% by weight, or from 65% to 95% by weight, relative to the total weight of the liquid fatty phase.

The at least one non-silicone-based liquid organic compound may be chosen from:
non-silicone-based liquid organic compounds having a global solubility parameter according to the Hansen solubility space of less than or equal to 18 (MPa)^{1/2}; and
monoalcohols having a global solubility parameter according to the Hansen solubility space of less than or equal to 20 (MPa)^{1/2}.

The non-silicone-based liquid fatty phase may thus optionally comprise at least one silicone-based liquid organic compound or oil, such as those mentioned above, which may be present in an amount of less than 50% by weight, such as an amount ranging from 0.1% to 40% by weight, ranging from 1% to 35% by weight, or ranging from 5% to 30% by weight, relative to the total weight of the liquid fatty phase.

According to one embodiment disclosed herein, the non-silicone-based liquid fatty phase does not contain any silicone-based liquid organic compounds or oils.

When the liquid fatty phase is a non-silicone-based liquid fatty phase, the macromonomers present in the grafted polymer may be carbon-based macromonomers as described below.

For example, when the liquid fatty phase is a non-silicone-based liquid fatty phase, the grafted polymer present in the composition may be a non-silicone grafted polymer.

As used herein, the term “non-silicone-based grafted polymer” means a grafted polymer predominantly containing a carbon-based macromonomer and optionally containing not more than 7% by weight, such as not more than 5% by weight, of silicone macromonomer, or even being free of silicone macromonomer.

According to another embodiment disclosed herein, the liquid fatty phase may be a silicone-based liquid fatty phase.

As used herein, the term “silicone-based liquid fatty phase” means a fatty phase comprising at least one silicone-based liquid organic compound or silicone oil such as those described above, the silicone compounds being predominantly present in the liquid fatty phase, i.e., present in an amount of at least 50% by weight, such as from 50% to 100% by weight, from 60% to 100%, from 60% to 99%, from 65% to 100%, or from 65% to 95% by weight, relative to the total weight of the liquid fatty phase.

The at least one silicone-based liquid organic compound may be chosen from:
liquid organic compounds, which may be non-silicone-based or silicone-based, with an overall solubility parameter according to the Hansen solubility space of less than or equal to 18 (MPa)^{1/2}.

The silicone-based liquid fatty phase may thus optionally comprise at least one non-silicone-based liquid organic compound or oil, as described above, which may be present in an amount of less than 50% by weight, such as an amount ranging from 0.1% to 40% by weight, from 1% to 35% by weight, or from 5% to 30% by weight, relative to the total weight of the liquid fatty phase.

According to one embodiment disclosed herein, the silicone-based liquid fatty phase does not contain any non-silicone-based liquid organic compounds.

When the liquid fatty phase is a silicone-based liquid fatty phase, the macromonomers present in the grafted polymer may be silicone-based macromonomers as described below.

For example, when the liquid fatty phase is a silicone-based liquid fatty phase, the grafted polymer present in the composition may be a silicone-based grafted polymer.

As used herein, the term “silicone-based grafted polymer” means a grafted polymer predominantly containing a silicone-based macromonomer and optionally containing up to 7% by weight, such as up to 5% by weight, of carbon-based macromonomer, or even being free of carbon-based macromonomer.

The choice of monomers constituting the skeleton of the polymer, of macromonomers, the molecular weight of the polymer, and the proportion of the monomers and macromonomers may be made as a function of the liquid organic dispersion medium so as to obtain a dispersion of particles of grafted polymers, for example a stable dispersion, it being possible for a person skilled in the art to make this choice.

As used herein, the term “stable dispersion” means a dispersion that is not liable to form a solid deposit or to undergo liquid/solid phase separation, for example after centrifugation, for example at 4,000 rpm for 15 minutes.

The grafted ethylenic polymer forming the particles in dispersion thus comprises a skeleton that is insoluble in the dispersion medium and a portion that is soluble in the dispersion medium.

The grafted ethylenic polymer may be a random polymer.

According to the present disclosure, the term “grafted ethylenic polymer” means a polymer that may be obtained by free-radical polymerization:
of at least one ethylenic monomer;
with at least one macromonomer, in an organic polymerization medium.

As used herein, the term “grafted acrylic polymer” means a polymer that may be obtained by free-radical polymerization:
of at least one acrylic monomer, and optionally of at least one additional non-acrylic vinyl monomer;
with at least one macromonomer, in an organic polymerization medium.

In certain embodiments, the acrylic monomers are present in an amount ranging from 50% to 100% by weight, such as from 55% to 100% by weight, from 55% to 95% by weight, from 60% to 100% by weight, or from 60% to 90% by weight, relative to the total weight of the mixture of acrylic monomers plus optional non-acrylic vinyl monomers.

In certain embodiments, the acrylic monomers are chosen from monomers whose homopolymer is insoluble in the dispersion medium under consideration, i.e., the homopolymer is in solid (or non-dissolved) form at a concentration of greater than or equal to 5% by weight at room temperature (20° C.) in the dispersion medium.

According to the present disclosure, the expression “macromonomer containing a polymerizable end group” means any polymer comprising on only one of its ends a polymerizable end group capable of reacting during the polymerization reaction with acrylic monomers and optionally the additional non-acrylic vinyl monomers constituting the skeleton. The macromonomer may make it possible to form the side chains of the grafted acrylic polymer. The polymerizable group of the macromonomer may be an ethylenically unsaturated group capable of free-radical polymerization with the monomers constituting the skeleton.

As used herein, the term “carbon-based macromonomer” means a non-silicone-based macromonomer, such as an oligomeric macromonomer obtained by polymerization of at least one ethylenically unsaturated non-silicone-based monomer, and mainly by polymerization of acrylic and/or non-acrylic vinyl monomers.

As used herein, the term “silicone-based macromonomer” means an organopolysiloxane macromonomer, such as a polydimethylsiloxane macromonomer.

In certain embodiments, the macromonomer is chosen from macromonomers whose homopolymer is soluble in the dispersion medium under consideration, i.e., fully dissolved at a concentration of greater than or equal to 5% by weight and at room temperature in the dispersion medium.

Thus, the grafted acrylic polymer comprises a skeleton (or main chain) comprising a sequence of acrylic units resulting from the polymerization, for example, of at least one acrylic monomer and of at least one side chain (or graft) derived from the reaction of the macromonomers, the at least one chain being covalently bonded to the main chain.

The skeleton (or main chain) is insoluble in the dispersion medium under consideration, whereas the at least one side chain (or graft) is soluble in the dispersion medium.

In the present disclosure, the term “acrylic monomers” means monomers chosen from (meth)acrylic acid, (meth)acrylic acid esters (also known as (meth)acrylates), and (meth)acrylic acid amides (also known as (meth)acrylamides).

As acrylic monomers that may be used to constitute the insoluble skeleton of the polymer, mention may be made of at least one of the following monomers, and also the salts thereof:
(i) the (meth)acrylates of formula: 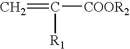
   in which:
R₁ is chosen from hydrogen and methyl groups;
R₂ is a group chosen from:
   linear or branched alkyl groups containing from 1 to 6 carbon atoms, the groups optionally comprising in their chains at least one hetero atom chosen from O, N, and S; and/or optionally comprising at least one substituent chosen from —OH, halogen atoms (F, Cl, Br, and I), and —NR′R″ wherein R′ and R″, which may be identical or different, are chosen from linear or branched C₁-C₄ alkyl groups; and/or optionally being substituted with at least one polyoxyalkylene group, such as with C₂-C₄ alkylene, for example polyoxyethylene and polyoxypropylene, the polyoxyalkylene group comprising a repetition of 5 to 30 oxyalkylene units;
   a cyclic alkyl group containing from 3 to 6 carbon atoms, the group optionally comprising in its chain at least one hetero atom chosen from O, N, and S, and/or optionally comprising at least one substituent chosen from OH and halogen atoms (F, Cl, Br, and I).

Examples of R₂ that may be mentioned include methyl, ethyl, propyl, butyl, isobutyl, methoxyethyl, ethoxyethyl, methoxypolyoxyethylene (350 OE), trifluoroethyl, 2-hydroxyethyl, 2-hydroxypropyl, dimethylaminoethyl, diethylaminoethyl, and dimethylaminopropyl groups;
(ii) (meth)acrylamides of formula: 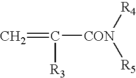
   in which:
R₃ is chosen from hydrogen and methyl groups;
R₄ and R₅, which may be identical or different, are chosen from hydrogen and linear or branched alkyl groups containing from 1 to 6 carbon atoms, optionally comprising at least one substituent chosen from —OH, halogen atoms (F, Cl, Br, and I), and —NR′R″, wherein R′ and R″, which may be identical or different, are chosen from linear or branched C₁-C₄ alkyls; or
R₄ is a hydrogen atom and R₅ is a 1,1-dimethyl-3-oxobutyl group.

As examples of alkyl groups that can constitute R₄ and R₅, mention may be made of n-butyl, t-butyl, n-propyl, dimethylaminoethyl, diethylaminoethyl, and dimethylaminopropyl;
(iii) (meth)acrylic monomers comprising at least one carboxylic acid, phosphoric acid, or sulfonic acid function, such as acrylic acid, methacrylic acid, and acrylamidopropanesulfonic acid.

Among these acrylic monomers, mention may be made of methyl, ethyl, propyl, butyl, and isobutyl (meth)acrylates; methoxyethyl (meth)acrylates, ethoxyethyl (meth)acrylates; trifluoroethyl methacrylate; dimethylaminoethyl methacrylate; diethylaminoethyl methacrylate; 2-hydroxypropyl methacrylate; 2-hydroxyethyl methacrylate; 2-hydroxypropyl acrylate; 2-hydroxyethyl acrylate; dimethylaminopropylmethacrylamide; and the salts thereof, and mixtures thereof.

In at least one embodiment, the acrylic monomers may be chosen from methyl acrylate, methoxyethyl acrylate, methyl methacrylate, 2-hydroxyethyl methacrylate, acrylic acid, and dimethylaminoethyl methacrylate, and mixtures thereof.

Among the additional non-acrylic vinyl monomers that may be mentioned are:
vinyl esters of formula: R₆—COO—CH═CH₂
   in which R₆ is chosen from linear or branched alkyl groups containing from 1 to 6 atoms and cyclic alkyl groups containing from 3 to 6 carbon atoms and/or an aromatic group, for example a benzene, anthracene, or naphthalene aromatic group;
non-acrylic vinyl monomers comprising at least one functional group chosen from carboxylic acid, phosphoric acid, and sulfonic acid functional groups, such as crotonic acid, maleic anhydride, itaconic acid, fumaric acid, maleic acid, styrenesulfonic acid, vinylbenzoic acid, vinylphosphoric acid, and the salts thereof; and
non-acrylic vinyl monomers comprising at least one tertiary amine functional group, such as 2-vinylpyridine and 4-vinylpyridine, and mixtures thereof.

In certain embodiments, the acrylic monomers present in the grafted polymer comprise at least (meth)acrylic acid and at least one monomer chosen from the (meth)acrylates and (meth)acrylamides described previously in points (i) and (ii). For example, the acrylic monomers may comprise at least (meth)acrylic acid and at least one monomer chosen from C₁-C₃ alkyl (meth)acrylates. (Meth)acrylic acid may be present in an amount of at least 5% by weight, such as an amount ranging from 5% to 80% by weight, an amount of at least 10% by weight, an amount ranging from 10% to 70% by weight, an amount of at least 15% by weight, or an amount ranging from 15% to 60% by weight, relative to the total weight of the polymer.

Among the salts that may be mentioned are those obtained by neutralization of acid groups with mineral bases such as sodium hydroxide, potassium hydroxide, and ammonium hydroxide, or organic bases such as alkanolamines, for instance monoethanolamine, diethanolamine, triethanolamine, and 2-methyl-2-amino-1-propanol.

Mention may also be made of the salts formed by neutralization of tertiary amine units, for example using a mineral or organic acid. Among the mineral acids that may be mentioned are sulfuric acid, hydrochloric acid, hydrobromic acid, hydriodic acid, phosphoric acid, and boric acid. Among the organic acids that may be mentioned are acids comprising at least one group chosen from carboxylic, sulfonic, and phosphonic groups. They may be chosen from linear, branched, or cyclic aliphatic acids and aromatic acids. These acids may also comprise at least one hetero atom chosen from O and N, for example in the form of hydroxyl groups. Acetic acid, propionic acid, terephthalic acid, citric acid, and tartaric acid may be mentioned.

According to one embodiment disclosed herein, the grafted ethylenic polymer contains no additional non-acrylic vinyl monomers as described above. In this embodiment, the insoluble skeleton of the grafted ethylenic polymer is formed solely from acrylic monomers as described above.

It is understood that these non-polymerized acrylic monomers may be soluble in the dispersion medium under consideration, but the polymer formed with these monomers is insoluble in the dispersion medium.

According to one embodiment disclosed herein, the grafted ethylenic polymer may be obtained by free-radical polymerization in an organic polymerization medium:
of at least one main acrylic monomer chosen from C₁-C₃ alkyl (meth)acrylates and optionally of at least one additional acrylic monomer chosen from (meth)acrylic acid, methacrylic acid and alkyl(meth)acrylates of formula (I) defined below, and salts thereof, to form the insoluble skeleton; and
of at least one silicone-based macromonomer comprising a polymerizable end group, as defined above.

Main acrylic monomers that may be used include methyl acrylate, methyl methacrylate, ethyl acrylate, ethyl methacrylate, n-propyl acrylate, n-propyl methacrylate, isopropyl acrylate, and isopropyl methacrylate, and mixtures thereof.

Methyl acrylate, methyl methacrylate, and ethyl methacrylate may, for example, be mentioned.

The additional acrylic monomers may be chosen from:
(meth)acrylic acid and its salts,
the (meth)acrylates of formula (I), and salts thereof: 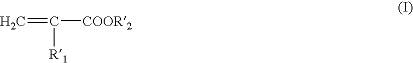
   in which:
R′₁ is chosen from hydrogen and methyl groups;
R′₂ is chosen from
a linear or branched alkyl group containing from 1 to 6 carbon atoms, the group comprising in its chain at least one oxygen atom and/or comprising at least one substituent chosen from OH, halogen atoms (F, Cl, Br, and I), and —NR′R″, wherein R′ and R″, which may be identical or different, are chosen from linear or branched C₁-C₃ alkyl groups;
a cyclic alkyl group containing from 3 to 6 carbon atoms, the group optionally comprising in its chain at least one oxygen atom and/or optionally comprising at least one substituent chosen from OH and halogen atoms (F, Cl, Br, and I);
   and mixtures thereof.

Examples of R₁₂ that may be mentioned include methoxyethyl, ethoxyethyl, trifluoroethyl, 2-hydroxyethyl, 2-hydroxypropyl, dimethylaminoethyl, diethylaminoethyl, and dimethylaminopropyl groups.

Among these additional acrylic monomers, mention may be made, for example, of (meth)acrylic acid, methoxyethyl (meth)acrylates, ethoxyethyl (meth)acrylates, trifluoroethyl methacrylate, dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate, and the salts thereof, and mixtures thereof.

Acrylic acid and methacrylic acid may be mentioned, for example.

The macromonomers comprise at one of the ends of the chain a polymerizable end group capable of reacting during the polymerization with the acrylic monomers and optionally the additional vinyl monomers, to form the side chains of the grafted ethylenic polymer. The polymerizable end group may be chosen from vinyl, (meth)acrylate, and (meth)acryloxy groups, such as (meth)acrylate groups.

The macromonomers may be chosen from macromonomers whose homopolymer has a glass transition temperature (Tg) of less than or equal to 25° C., for example ranging from −100° C. to 25° C. or ranging from −80° C. to 0° C.

The macromonomers have a weight-average molar mass of greater than or equal to 200, such as greater than or equal to 300, greater than or equal to 500, or greater than 600.

The macromonomers may have a weight-average molar mass (Mw) ranging from 200 to 100,000, such as ranging from 500 to 50,000, ranging from 800 to 20,000, ranging from 800 to 10,000, or ranging from 800 to 6,000.

In the present disclosure, the weight-average (Mw) and number-average (Mn) molar masses are determined by liquid gel permeation chromatography (THF solvent, calibration curve established with linear polystyrene standards, refractometric detector).

Carbon-based macromonomers that may be mentioned include:
(i) homopolymers and copolymers of linear or branched C₈-C₂₂ alkyl acrylate or methacrylate, containing a polymerizable end group chosen from at least one of vinyl and (meth)acrylate groups, among which mention may be made of: poly(2-ethylhexyl acrylate) macromonomers with a mono(meth)acrylate end group; poly(dodecyl acrylate) or poly(dodecyl methacrylate) macromonomers with a mono(meth)acrylate end group; and poly(stearyl acrylate) or poly(stearyl methacrylate) macromonomers with a mono(meth)acrylate end group.

Such macromonomers are described, for example, in European Patent Nos. EP 895 467 and EP 96459, and in the article by Gillman K. F., Polymer Letters, Vol 5, page 477-481 (1967).

With respect to at least one embodiment, mention may be made of macromonomers based on poly(2-ethylhexyl acrylate) or poly(dodecyl acrylate) with a mono(meth)acrylate end group;
(ii) polyolefins containing an ethylenically unsaturated end group, for example containing a (meth)acrylate end group. Examples of such polyolefins that may be mentioned include the following macromonomers, it being understood that they have a (meth)acrylate end group: polyethylene macromonomers, polypropylene macromonomers, macromonomers of polyethylene/polypropylene copolymer, macromonomers of polyethylene/polybutylene copolymer, polyisobutylene macromonomers, polybutadiene macromonomers, polyisoprene macromonomers, polybutadiene macromonomers, and poly(ethylene/butylene)-polyisoprene macromonomers.

Such macromonomers are described, for example, in U.S. Pat. No. 5,625,005, which mentions ethylene/butylene and ethylene/propylene macromonomers containing a (meth)acrylate reactive end group.

Mention may be made, with respect to at least one embodiment, of the poly(ethylene/butylene) methacrylate such as that sold under the name Kraton Liquid® L-1253 by Kraton Polymers.

Silicone-based macromonomers that may be mentioned include polydimethylsiloxanes containing mono(meth)acrylate end groups, for example those of formula (II) below: 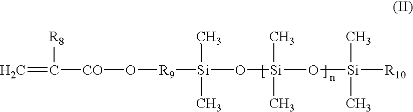
in which
R₈ is chosen from hydrogen and methyl groups;
R₉ is a divalent hydrocarbon-based group containing from 1 to 10 carbon atoms and optionally containing one or two ether bonds —O—;
R₁₀ is an alkyl group containing from 1 to 10 carbon atoms, such as from 2 to 8 carbon atoms;
n is an integer ranging from 1 to 300, for example, ranging from 3 to 200 or from 5 to 100.

Silicone-based macromonomers that may be used include monomethacryloxypropyl polydimethylsiloxanes such as those sold under the name PS560-K6 by the company United Chemical Technologies Inc. (UCT) and under the name MCR-M17 by the company Gelest Inc.

In certain embodiments, the polymerized macromonomer (comprising the side chains of the grafted polymer) is present in an amount ranging from 0.1% to 15% by weight, such as from 0.2% to 10% by weight or from 0.3% to 8% by weight, relative to the total weight of the polymer.

As grafted ethylenic polymer dispersed in a non-silicone-based liquid fatty phase, mention may be made, with respect to at least one embodiment, of those obtained by polymerization:
of methyl acrylate and of a polyethylene/polybutylene macromonomer containing a methacrylate end group (such as Kraton® L-1253), for example in a solvent chosen from isododecane, isononyl isononanoate, octyldodecanol, diisostearyl malate, and C₁₂-C₁₅ alkyl benzoate (such as Finsolv® TN);
of methoxyethyl acrylate and of a polyethylene/polybutylene macromonomer containing a methacrylate end group (such as Kraton® L-1253), for example in isododecane;
of methyl acrylate/methyl methacrylate monomers and of a polyethylene/polybutylene macromonomer containing a methacrylate end group (such as Kraton® L-1253), for example in isododecane;
of methyl acrylate/acrylic acid monomers and of a polyethylene/polybutylene macromonomer containing a methacrylate end group (such as Kraton® L-1253), for example in isododecane;
of methyl acrylate/dimethylaminoethyl methacrylate monomers and of a polyethylene/polybutylene macromonomer containing a methacrylate end group (such as Kraton® L-1253), for example in isododecane;
of methyl acrylate/2-hydroxyethyl methacrylate monomers and of a polyethylene/polybutylene macromonomer containing a methacrylate end group (such as Kraton® L-1253), for example in isododecane.

As grafted acrylic polymer dispersed in a silicone-based liquid fatty phase, mention may be made of those obtained by polymerization:
of methyl acrylate and of the monomethacryloyloxypropyl polydimethylsiloxane macromonomer with a weight-average molecular weight ranging from 800 to 6,000, for example in decamethylcyclopentasiloxane and phenyl trimethicone;
of methyl acrylate, acrylic acid, and the monomethacryloxypropyl polydimethylsiloxane macromonomer with a weight-average molecular weight ranging from 800 to 6,000, for example decamethylcyclopentasiloxane and phenyl trimethicone.

The weight-average molar mass (Mw) of the grafted polymer may range from 10,000 to 300,000, such as from 20,000 to 200,000, for example, from 25,000 to 150,000.

By virtue of the above-mentioned characteristics, in a given organic dispersion medium, the polymers have the capacity of folding over on themselves, thus forming particles of substantially spherical shape, the periphery of these particles having the deployed side chains, which may ensure the stability of these particles. Such particles resulting from the characteristics of the grafted polymer may have the particular feature of not aggregating in the medium and thus of being self-stabilized and of forming a particularly stable polymer particle dispersion.

For example, the grafted ethylenic polymers of the dispersion may be capable of forming nanometer-sized particles, with a mean size ranging from 10 to 400 nm, such as from 20 to 200 nm.

As a result of this small size, the grafted polymer particles in dispersion may be stable and therefore have little susceptibility to form aggregates.

The dispersion of grafted polymer may thus be a dispersion that is stable and does not form sediments when it is placed at room temperature (25° C.) for an extended period (for example 24 hours).

In certain embodiments, the dispersion of grafted polymer particles has a solids content (or dry extract) of polymer ranging from 40% to 70% by weight of solids, such as from 45% to 65% by weight.

The dispersion of grafted polymer particles may be prepared via a process comprising a free-radical copolymerization step, in an organic polymerization medium, of at least one acrylic monomer as defined above with at least one macromonomer as defined above.

As mentioned above, the liquid organic dispersion medium may be identical to or different from the polymerization medium.

The copolymerization may be performed conventionally in the presence of a polymerization initiator. The polymerization initiators may be free-radical initiators. In general, such a polymerization initiator may be chosen from organic peroxide compounds such as dilauroyl peroxide, dibenzoyl peroxide, and tert-butyl peroxy-2-ethylhexanoate; and diazo compounds such as azobisisobutyronitrile and azobisdimethylvaleronitrile.

The reaction may also be initiated using photoinitiators, radiation such as UV or neutrons; or plasma.

In general, to perform this process, at least a portion of the organic polymerization medium, a portion of the additional acrylic and/or vinyl monomers, which will constitute the insoluble skeleton after polymerization, all of the macromonomer (which will constitute the side chains of the polymer), and a portion of the polymerization initiator are introduced into a reactor whose size is suitable for the amount of polymer to be prepared. At this stage of introduction, the reaction medium may form a relatively homogeneous medium.

The reaction medium is then stirred and heated up to a temperature to obtain polymerization of the monomers and macromonomers. After a certain time, the initially homogeneous and clear medium leads to a dispersion of milky appearance. A mixture comprising the remaining portion of monomers and of polymerization initiator is then added. After an adequate time during which the mixture is heated with stirring, the medium stabilizes in the form of a milky dispersion, the dispersion comprising polymer particles stabilized in the medium in which they have been created, the stabilization being due to the presence, in the polymer, of side chains that are soluble in the dispersion medium.

The grafted polymer may be present in the composition according to the invention in a solids content (or active material content) ranging from 1% to 70% by weight, such as from 5% to 60% by weight, from 6% to 45% by weight, or from 8% to 40% by weight, relative to the total weight of the composition.

The composition disclosed herein may contain, as film-forming polymer, a linear block ethylenic polymer, referred to hereinbelow as a “block polymer”, the structure of which is described below.

As used herein, the term “block” polymer means a polymer comprising at least two different blocks, for example comprising at least three different blocks.

The block polymer is a polymer of linear structure. In contrast, a polymer of non-linear structure is, for example, a polymer of branched, star, or grafted structure, or the like.

In certain embodiments, the block polymer is free of styrene. As used herein, the term “polymer free of styrene” means a polymer containing less than 10% by weight, such as less than 5% by weight, less than 2% by weight, or less than 1% by weight of styrene monomer, for instance styrene; styrene derivatives such as methylstyrene, chlorostyrene, and chloromethylstyrene; and even polymer containing no styrene monomer, relative to the total weight of the polymer.

In certain embodiements, the block polymer comprises at least one first block and at least one second block that have different glass transition temperatures (Tg), the first and second blocks being linked together via an intermediate block comprising at least one constituent monomer of the first block and at least one constituent monomer of the second block.

As used herein, the term “at least one block” means one or more blocks.

The intermediate block is a block comprising at least one constituent monomer of the first block and at least one constituent monomer of the second block of the polymer allowing these blocks to be “compatibilized”.

It is pointed out that, in the present disclosure, the terms “first” and “second” blocks do not in any way condition the order of the blocks in the structure of the block polymer.

In certain embodiments, the first and second blocks of the block polymer are mutually incompatible.

As used herein, the term “mutually incompatible blocks” means that the mixture formed from the polymer corresponding to the first block and of the polymer corresponding to the second block is not miscible in the organic liquid that is in major amount by weight contained in the liquid fatty phase, at room temperature (25° C.) and atmospheric pressure (10⁵ Pa), for a content of the polymer mixture of greater than or equal to 5% by weight, relative to the total weight of the mixture (polymers and solvent), it being understood that:
i) the polymers are present in the mixture in an amount such that the respective weight ratio ranges from 10/90 to 90/10, and that
ii) each of the polymers corresponding to the first and second blocks has an average (weight-average or number-average) molar mass equal to that of the block polymer±15%.

When the liquid fatty phase comprises a mixture of organic liquids, in the case of two or more organic liquids present in identical mass proportions, the polymer mixture is immiscible in at least one of them.

Needless to say, in the case where the liquid fatty phase comprises only one organic liquid, this liquid is the predominant organic liquid.

In certain embodiments, the block polymer comprises no silicon atoms in its skeleton. The term “skeleton” means the main chain of the polymer, as opposed to the pendent side chains.

In certain embodiments, the block polymer is not soluble in water or in a mixture of water and linear or branched lower monoalcohols containing from 2 to 5 carbon atoms, for instance ethanol, isopropanol, and n-propanol, without modifying the pH, at an active material content of at least 1% by weight, at room temperature (25° C.).

In certain embodiments, the block polymer is not an elastomer.

As used herein, the term “non-elastomeric polymer” means a polymer which, when it is subjected to a constraint intended to stretch it (for example by 30% relative to its initial length), does not return to a length substantially identical to its initial length when the constraint ceases.

More specifically, the term “non-elastomeric polymer” denotes a polymer with an instantaneous recovery Rᵢ<50% and a delayed recovery R₂ₕ<70% after having been subjected to a 30% elongation. For example, R₁ may be <30% and R₂ₕ may be <50%.

More specifically, the non-elastomeric nature of the polymer may be determined according to the following protocol:

A polymer film is prepared by pouring a solution of the polymer in a Teflon®-coated mold, followed by drying for 7 days in an environment conditioned at 23±5° C. and 50±10% relative humidity.

A film about 100 μm thick is thus obtained, from which are cut rectangular specimens (for example using a punch) 15 mm wide and 80 mm long.

This sample is subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (l₀) of the specimen.

The instantaneous recovery R₁ may be determined in the following manner:
the specimen is pulled by 30% (εₘₐₓ), i.e., about 0.3 times its initial length (l₀)
the constraint is released by applying a return speed equal to the tensile speed, i.e., 50 mm/min, and the residual elongation of the specimen is measured as a percentage, after returning to zero constraint (εᵢ).

The percentage instantaneous recovery (Rᵢ) is given by the following formula:
Rᵢ=(εₘₐₓ−εᵢ)/εₘₐₓ)×100

To determine the delayed recovery, the percentage residual elongation of the specimen (ε₂ₕ) is measured 2 hours after returning to zero constraint.

The percentage delayed recovery (R₂ₕ) is given by the following formula:
R₂ₕ=(εₘₐₓ−ε₂ₕ)/εₘₐₓ)×100

Purely as a guide, a polymer according to one embodiment disclosed herein may have an instantaneous recovery Rᵢ of 10% and a delayed recovery R₂ₕ of 30%.

In certain embodiments, the block polymer has a polydispersity index I of greater than 2, for example ranging from 2 to 9, greater than or equal to 2.5, ranging from 2.5 to 8, greater than or equal to 2.8, or ranging from 2.8 to 6.

The polydispersity index I of the block polymer is equal to the ratio of the weight-average mass Mw to the number-average mass Mn.

The weight-average molar mass (Mw) and number-average molar mass (Mn) may be determined by gel permeation liquid chromatography (THF solvent, calibration curve established with linear polystyrene standards, refractometric detector).

The weight-average mass (Mw) of the block polymer may be less than or equal to 300,000. It may range, for example, from 35,000 to 200,000, such as from 45,000 to 150,000.

The number-average mass (Mn) of the block polymer may be less than or equal to 70,000. It may range, for example, from 10,000 to 60,000, such as from 12,000 to 50,000.

Each block of the block polymer is derived from one type of monomer or from several different types of monomer.

This means that each block may comprise a homopolymer or a copolymer; this copolymer constituting the block may in turn be random or alternating.

In certain embodiments, the intermediate block comprising at least one constituent monomer of the first block and at least one constituent monomer of the second block of the block polymer is a random polymer.

In certain embodiments, the intermediate block is derived essentially from constituent monomers of the first block and of the second block.

As used herein, the term “essentially” means at least 85%, such as at least 90%, for example, at least 95%, or even 100%.

In certain embodiments, the intermediate block has a glass transition temperature Tg that is between the glass transition temperature of the first block and the glass transition temperature of the second block.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:
1/Tg=Σᵢ(\overline{ω}ᵢ/Tgᵢ),
\overline{ω}ᵢ being the mass fraction of the monomer i in the block under consideration and Tgᵢ being the glass transition temperature of the homopolymer of the monomer i.

Unless otherwise indicated, the Tg values indicated for the first and second blocks in the present disclosure are theoretical Tg values.

The difference between the glass transition temperatures of the first and second blocks may be greater than 10° C., such as greater than 20° C. or greater than 30° C.

For example, the first block of the block polymer may be chosen from:
a) blocks with a Tg of greater than or equal to 40° C.,
b) blocks with a Tg of less than or equal to 20° C., and
c) blocks with a Tg of between 20 and 40° C.;
and the second block may be chosen from categories a), b), and c), different from the first block.

As used herein, the expression “between . . . and . . . ” is intended to denote a range of values for which the limits mentioned are excluded, and “from . . . to . . . ” and “ranging from . . . to . . . ” are intended to denote a range of values for which the limits are included.

a) Blocks with a Tg of Greater Than or Equal to 40° C.

Blocks with a Tg of greater than or equal to 40° C. may have, for example, a Tg ranging from 40 to 150° C., such as a Tg greater than or equal to 50° C., for example ranging from 50° C. to 120° C. or greater than or equal to 60° C., for example ranging from 60° C. to 120° C.

Blocks with a Tg of greater than or equal to 40° C. may be a homopolymer or a copolymer.

In the case where the block is a homopolymer, it may be derived from monomers which are such that the homopolymers prepared from these monomers have glass transition temperatures of greater than or equal to 40° C. This first block may be a homopolymer comprising only one type of monomer (for which the Tg of the corresponding homopolymer is greater than or equal to 40° C.).

In the case where the first block is a copolymer, it may be totally or partially derived from at least one monomer, the nature and concentration of which are chosen such that the Tg of the resulting copolymer is greater than or equal to 40° C. The copolymer may comprise, for example:
monomers which are such that the homopolymers prepared from these monomers have Tg values of greater than or equal to 40° C., for example a Tg ranging from 40 to 150° C., a Tg greater than or equal to 50° C., for example ranging from 50° C. to 120° C., or a Tg greater than or equal to 60° C., for example ranging from 60° C. to 120° C., and
monomers which are such that the homopolymers prepared from these monomers have Tg values of less than 40° C., chosen from monomers with a Tg of between 20 and 40° C. and/or monomers with a Tg of less than or equal to 20° C., for example a Tg ranging from −100 to 20° C., for example less than 15° C., a Tg ranging from −80° C. to 15° C., a Tg less than 10° C., or a Tg ranging from −50° C. to 0° C., as described below.

The monomers whose homopolymers have a glass transition temperature of greater than or equal to 40° C. may be chosen from at least one of the following monomers, also known as the main monomers:
methacrylates of formula CH₂═C(CH₃)—COOR₁
   in which R₁ is a linear or branched unsubstituted alkyl group containing from 1 to 4 carbon atoms, such as a methyl, ethyl, propyl, and isobutyl groups or R₁ is a C₄ to C₁₂ cycloalkyl group,
acrylates of formula CH₂═CH—COOR₂
   in which R₂ is a C₄ to C₁₂ cycloalkyl group such as isobornyl acrylate or a tert-butyl group, and
(meth)acrylamides of formula: 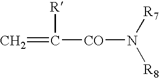
   in which R₇ and R₈, which may be identical or different, are chosen from hydrogen and linear or branched C₁ to C₁₂ alkyl groups, such as an n-butyl, t-butyl, isopropyl, isohexyl, isooctyl, and isononyl groups; or R₇ is hydrogen and R₈ is a 1,1-dimethyl-3-oxobutyl group, and R′ is chosen from hydrogen and methyl groups. Examples of monomers that may be mentioned include N-butylacrylamide, N-t-butylacrylamide, N-isopropylacrylamide, N,N-dimethylacrylamide, and N,N-dibutylacrylamide.

Main monomers that may be mentioned with respect to at least one embodiment are at least one of methyl methacrylate, isobutyl (meth)acrylate, and isobornyl (meth)acrylate.

b) Blocks with a Tg of Less Than or Equal to 20° C.

Blocks with a Tg of less than or equal to 20° C. may have, for example, a Tg ranging from −100 to 20° C., such as less than or equal to 15° C., for example a Tg ranging from −80° C. to 15° C., or a Tg less than or equal to 10° C., for example ranging from −50° C. to 0° C.

Blocks with a Tg of less than or equal to 20° C. may be a homopolymer or a copolymer.

In the case where the block is a homopolymer, it may be derived from monomers which are such that the homopolymers prepared from these monomers have glass transition temperatures of less than or equal to 20° C. This second block may be a homopolymer comprising only one type of monomer (for which the Tg of the corresponding homopolymer is less than or equal to 20° C.).

In the case where the block with a Tg of less than or equal to 20° C. is a copolymer, it may be totally or partially derived from at least one monomer, the nature and concentration of which are chosen such that the Tg of the resulting copolymer is less than or equal to 20° C.

It may comprise, for example
at least one monomer whose corresponding homopolymer has a Tg of less than or equal to 20° C., for example a Tg ranging from −100° C. to 20° C., such as a Tg less than 150° C., for example ranging from −80° C. to 150° C., or a Tg less than 10° C., for example ranging from −50° C. to 0° C., and
at least one monomer whose corresponding homopolymer has a Tg of greater than 20° C., such as monomers with a Tg of greater than or equal to 40° C., for example a Tg ranging from 40 to 150° C., a Tg greater than or equal to 50° C., for example ranging from 50° C. to 120° C., or a Tg greater than or equal to 60° C., for example ranging from 60° C. to 120° C. and/or monomers with a Tg of between 20 and 40° C., as described above.

In certain embodiments, the block with a Tg of less than or equal to 20° C. is a homopolymer.

The monomers whose homopolymer has a Tg of less than or equal to 20° C. may be chosen, for example, from the following monomers, or main monomers:
acrylates of formula CH₂═CHCOOR₃, wherein R₃ is a linear or branched C, to C₁₂ unsubstituted alkyl group, with the exception of the tert-butyl group, in which at least one hetero atom chosen from O, N, and S is optionally intercalated,
methacrylates of formula CH₂═C(CH₃)—COOR₄, wherein R₄ is a linear or branched C₆ to C₁₂ unsubstituted alkyl group, in which at least one hetero atom chosen from O, N, and S is optionally intercalated;
vinyl esters of formula R₅—CO—O—CH═CH₂ wherein R₅ is a linear or branched C₄ to C₁₂ alkyl group;
C₄ to C₁₂ alkyl vinyl ethers and alkyl ethers, and
N—(C₄ to C₁₂)alkyl acrylamides, such as N-octylacrylamide,
and mixtures thereof.

The main monomers that may be mentioned for the block with a Tg of less than or equal to 20° C. are alkyl acrylates whose alkyl chain contains from 1 to 10 carbon atoms, with the exception of the tert-butyl group, such as at least one of methyl acrylate, isobutyl acrylate, and 2-ethylhexyl acrylate.

c) Blocks with a Tg of Between 20 and 40° C.

Blocks with a Tg of between 20 and 40° C. may be a homopolymer or a copolymer.

In the case where the block is a homopolymer, it may be derived from monomers (or main monomers) which are such that the homopolymers prepared from these monomers have glass transition temperatures of between 20 and 40° C. This first block may be a homopolymer, comprising only one type of monomer (for which the Tg of the corresponding homopolymer ranges from 20° C. to 40° C.).

The monomers whose homopolymer has a glass transition temperature of between 20 and 40° C. may be chosen from n-butyl methacrylate, cyclodecyl acrylate, neopentyl acrylate, and isodecylacrylamide, and mixtures thereof.

In the case where the block with a Tg of between 20 and 40° C. is a copolymer, it is totally or partially derived from at least one monomer (or main monomer) whose nature and concentration are chosen such that the Tg of the resulting copolymer is between 20 and 40° C.

In certain embodiments, the block with a Tg of between 20 and 40° C. is a copolymer totally or partially derived from:
main monomers whose corresponding homopolymer has a Tg of greater than or equal to 40° C., for example a Tg ranging from 40° C. to 150° C., such as a Tg greater than or equal to 50° C., for example ranging from 50 to 120° C., or a Tg greater than or equal to 60° C., for example ranging from 60° C. to 120° C., as described above, and/or
main monomers whose corresponding homopolymer has a Tg of less than or equal to 20° C., for example a Tg ranging from −100 to 20° C., such as a Tg less than or equal to 15° C., for example ranging from −80° C. to 15° C., or a Tg less than or equal to 10° C., for example ranging from −50° C. to 0° C., as described above, the monomers being chosen such that the Tg of the copolymer forming the first block is between 20 and 40° C.

Such main monomers are chosen, for example, from methyl methacrylate, isobornyl acrylate, isobornyl methacrylate, butyl acrylate, and 2-ethylhexyl acrylate, and mixtures thereof.

In certain embodiments, the proportion of the second block with a Tg of less than or equal to 20° C. ranges from 10% to 85% by weight, such as from 20% to 70% or from 20% to 50% by weight of the polymer.

However, each of the blocks may contain in a small proportion at least one constituent monomer of the other block.

Thus, the first block may contain at least one constituent monomer of the second block, and vice versa.

Each of the first and/or second blocks of the block polymer may comprise, in addition to the monomers indicated above, at least one other monomer known as “additional monomers,” which are different from the main monomers mentioned above.

The nature and amount of this at least one additional monomer are chosen such that the block in which it is present has the desired glass transition temperature.

This at least one additional monomer may be chosen, for example, from hydrophilic monomers such as:
ethylenically unsaturated monomers comprising at least one carboxylic or sulfonic acid function, for instance: acrylic acid, methacrylic acid, crotonic acid, maleic anhydride, itaconic acid, fumaric acid, maleic acid, acrylamidopropanesulfonic acid, vinylbenzoic acid, vinylphosphoric acid, and salts thereof,
ethylenically unsaturated monomers comprising at least one tertiary amine function, for instance 2-vinylpyridine, 4-vinylpyridine, dimethylaminoethyl methacrylate, diethylaminoethyl methacrylate, dimethylaminopropylmethacrylamide, and salts thereof,
methacrylates of formula CH₂═C(CH₃)—COOR₆ in which R₆ is a linear or branched alkyl group containing from 1 to 4 carbon atoms, such as methyl, ethyl, propyl, and isobutyl groups, the alkyl group being substituted with at least one substituent chosen from hydroxyl groups (for instance 2-hydroxypropyl methacrylate and 2-hydroxyethyl methacrylate) and halogen atoms (Cl, Br, I, and F), such as trifluoroethyl methacrylate,
methacrylates of formula CH₂═C(CH₃)—COOR₉,
   wherein R₉ is a linear or branched C₆ to C₁₂ alkyl group in which at least one hetero atom chosen from O, N, and S is optionally intercalated, the alkyl group being substituted with at least one substituent chosen from hydroxyl groups and halogen atoms (Cl, Br, I, and F);
acrylates of formula CH₂═CHCOOR₁₀, wherein R₁₀ is a linear or branched C₁ to C₁₂ alkyl group substituted with at least one substituent chosen from hydroxyl groups and halogen atoms (Cl, Br, I, and F), such as 2-hydroxypropyl acrylate and 2-hydroxyethyl acrylate, or R₁₀ is a C₁ to C₁₂ alkyl-O—POE (polyoxyethylene) with repetition of the oxyethylene unit 5 to 30 times, for example methoxy-POE, or R₁₀ is a polyoxyethylenated group comprising from 5 to 30 ethylene oxide units; and ethylenically unsaturated monomers comprising at least one silicon atom, such as methacryloxypropyltrimethoxysilane and methacryloxypropyltris(trimethylsiloxy)silane.

Additional monomers that may be mentioned are at least one of acrylic acid, methacrylic acid, and trifluoroethyl methacrylate.

According to one embodiment disclosed herein, the block polymer is a non-silicone polymer, i.e., a polymer free of silicon atoms.

This at least one additional monomer may be present in an amount of less than or equal to 30% by weight, for example from 1% to 30% by weight, from 5% to 20% by weight, or from 7% to 15% by weight, relative to the total weight of the first and/or second blocks.

In certain embodiments, each of the first and second blocks comprises at least one monomer chosen from (meth)acrylic acid esters, and optionally at least one monomer chosen from (meth)acrylic acid.

In certain embodiments, each of the first and second blocks of the block polymer is totally derived from at least one monomer chosen from acrylic acid and (meth)acrylic acid esters, and optionally at least one monomer chosen from (meth)acrylic acid.

The block polymer may be obtained by free-radical solution polymerization according to the following preparation process:
a portion of the polymerization solvent is introduced into a suitable reactor and heated until the adequate temperature for the polymerization is reached (typically between 60 and 120° C.),
once this temperature is reached, the constituent monomers of the first block are introduced in the presence of part of the polymerization initiator,
after a time T corresponding to a maximum degree of conversion of 90%, the constituent monomers of the second block and the rest of the initiator are introduced,
the mixture is left to react for a time T′ (ranging from 3 to 6 hours), after which the mixture is cooled to room temperature,
the polymer dissolved in the polymerization solvent is obtained.

As used herein, the term “polymerization solvent” means a solvent or a mixture of solvents. In at least one embodiment, the polymerization solvent may be chosen from ethyl acetate; butyl acetate; alcohols such as isopropanol and ethanol; and aliphatic alkanes such as isododecane, and mixtures thereof. For example, the polymerization solvent may be a mixture of butyl acetate, isopropanol, and/or isododecane.

According to one embodiment, the block polymer comprises a first block with a Tg of greater than or equal to 40° C., as described above in a) and a second block with a Tg of less than or equal to 20° C., as described above in b).

In certain embodiments, the first block with a Tg of greater than or equal to 40° C. is a copolymer derived from monomers which are such that the homopolymer prepared from these monomers has a glass transition temperature of greater than or equal to 40° C., such as the monomers described above.

In another embodiment, the second block with a Tg of less than or equal to 20° C. is a homopolymer derived from monomers which are such that the homopolymer prepared from these monomers has a glass transition temperature of less than or equal to 20° C., such as the monomers described above.

In yet another embodiment, the proportion of the block with a Tg of greater than or equal to 40° C. ranges from 20% to 90%, such as from 30% to 80% or from 50% to 70% by weight, relative to the total weight of the polymer.

For example, the proportion of the block with a Tg of less than or equal to 20° C. may range from 5% to 75%, such as from 15% to 50% or from 25% to 45% by weight, relative to the total weight of the polymer.

According to the present disclosure, the block polymer may comprise:
a first block with a Tg of greater than or equal to 40° C., for example ranging from 85° C. to 115° C., which is an isobornyl acrylate/isobutyl methacrylate copolymer,
a second block with a Tg of less than or equal to 20° C., for example ranging from −85° C. to −55° C., which is a 2-ethylhexyl acrylate homopolymer, and
an intermediate block, which is an isobornyl acrylate/isobutyl methacrylate/2-ethylhexyl acrylate random copolymer.

According to another embodiment, the block polymer comprises a first block having a glass transition temperature (Tg) of between 20 and 40° C., in accordance with the blocks described in c) and a second block having a glass transition temperature of less than or equal to 20° C., as described above in b) or a glass transition temperature of greater than or equal to 40° C., as described in a) above.

In certain embodiments, the proportion of the first block with a Tg of between 20 and 40° C. ranges from 10% to 85%, such as from 30% to 80% or from 50% to 70% by weight, relative to the total weight of the polymer.

When the second block is a block with a Tg of greater than or equal to 40° C., it may be present in an amount ranging from 10% to 85% by weight, such as from 20% to 70% or from 30% to 70% by weight, relative to the total weight of the polymer.

When the second block is a block with a Tg of less than or equal to 20° C., it may be present in an amount ranging from 10% to 85% by weight, such as from 20% to 70% or from 20% to 50% by weight, relative to the total weight of the polymer.

In certain embodiments, the first block with a Tg of between 20 and 40° C. may be a copolymer derived from monomers which are such that the corresponding homopolymer has a Tg of greater than or equal to 40° C., and from monomers which are such that the corresponding homopolymer has a Tg of less than or equal to 20° C.

The second block with a Tg of less than or equal to 20° C. or with a Tg of greater than or equal to 40° C. may be a homopolymer.

According to one embodiment, the block polymer comprises:
a first block with a Tg of between 20 and 40° C., for example with a Tg of 21 to 39° C., which is a copolymer comprising isobornyl acrylate/isobutyl methacrylate/2-ethylhexyl acrylate,
a second block with a Tg of less than or equal to 20° C., for example ranging from −65 to −35° C., which is a homopolymer of methyl methacrylate, and
an intermediate block which is an isobornyl acrylate/isobutyl methacrylate/2-ethylhexyl acrylate random copolymer.

According to another embodiment, the block polymer may comprise:
a first block with a Tg of greater than or equal to 40° C., for example ranging from 85 to 115° C., which is an isobornyl methacrylate/isobutyl methacrylate copolymer,
a second block with a Tg of less than or equal to 20° C., for example ranging from −35 to −5° C., which is an isobutyl acrylate homopolymer, and
an intermediate block, which is an isobornyl methacrylate/isobutyl methacrylate/isobutyl acrylate random copolymer.

According to yet another embodiment, the block polymer may comprise:
a first block with a Tg of greater than or equal to 40° C., for example ranging from 60 to 90° C., which is an isobornyl acrylate/isobutyl methacrylate copolymer,
a second block with a Tg of less than or equal to 20° C., for example ranging from −35 to 5° C., which is an isobutyl acrylate homopolymer, and
an intermediate block, which is an isobornyl acrylate/isobutyl methacrylate/isobutyl acrylate random copolymer.

In one embodiment, the at least one film-forming polymer is an organic film-forming polymer that is soluble in the liquid fatty phase.

When the liquid fatty phase of the composition comprises at least one oil, the at least one film-forming polymer may be a polymer that is soluble in the oil. In this case, it may be referred to as a liposoluble polymer. The liposoluble polymer may be of any chemical type and may, for example, be chosen from:
a) liposoluble, amorphous homopolymers and copolymers of olefins, of cycloolefins, of butadiene, of isoprene, of styrene, of vinyl ethers, of vinyl esters, of vinyl amides, and of (meth)acrylic acid esters or amides comprising a linear, branched or cyclic C₄₋₅₀ alkyl group and which may be amorphous. The liposoluble homopolymers and copolymers that may be mentioned may be obtained from monomers chosen from at least one of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, and stearyl (meth)acrylate. Examples that may be mentioned include the alkyl acrylate/cycloalkyl acrylate copolymer sold by Phoenix Chem. under the name Giovarez® AC-5099 ML, and vinylpyrrolidone copolymers, such as copolymers of a C₂-C₃₀, for example C₃ to C₂₂, alkene, and combinations thereof, may be used. As examples of VP copolymers that may be used, mention may be made of copolymers of VP/vinyl laurate, VP/vinyl stearate, butylated polyvinylpyrrolidone (PVP), VP/hexadecene, VP/triacontene, and VP/acrylic acid/lauryl methacrylate.

Liposoluble copolymers that may be mentioned include:
i) acrylic-silicone grafted polymers comprising a silicone skeleton and acrylic grafts or comprising an acrylic skeleton and silicone grafts, such as the product sold under the name SA 70.5 by 3M and described in U.S. Pat. Nos. 5,725,882; 5,209,924; 4,972,037; 4,981,903; 4,981,902; 5,468,477; and 5,219,560 and in European Patent No. EP 0 388 582;
ii) liposoluble polymers belonging to one of the classes described above and bearing fluoro groups, such as those described in U.S. Pat. No. 5,948,393 and the alkyl (meth)acrylate/perfluoroalkyl (meth)acrylate copolymers described in European Patent No. EP 0 815 836 and U.S. Pat. No. 5,849,318; and
iii) polymers or copolymers resulting from the polymerization or copolymerization of an ethylenic monomer, comprising at least one ethylenic bond, which may be conjugated (or diene). As polymers or copolymers resulting from the polymerization or copolymerization of an ethylenic monomer, it is possible to use vinyl, acrylic, or methacrylic copolymers.

In one embodiment, the at least one film-forming polymer is a block copolymer comprising at least one block comprising styrene units or styrene derivatives (for example methylstyrene, chlorostyrene, and chloromethylstyrene). The copolymer comprising at least one styrene block may be chosen from diblock and triblock copolymers, and even multiblock copolymers, in starburst or radial form. The copolymer comprising at least one styrene block may also comprise, for example, at least one of alkylstyrene (AS) blocks, ethylene/butylene (EB) blocks, ethylene/propylene (EP) blocks, butadiene (B) blocks, isoprene (I) blocks, acrylate (A) blocks, and methacrylate (MA) blocks. The copolymer comprising at least one block comprising styrene units or styrene derivatives may be chosen from diblock and triblock copolymers, such as of the polystyrene/polyisoprene and polystyrene/polybutadiene type, such as those sold or manufactured under the name Luvitol® HSB by BASF, and those of the polystyrene/copoly(ethylene-propylene) type or alternatively of the polystyrene/copoly(ethylene-butylene) type, such as those sold or manufactured under the brand name Kraton® by Shell Chemical Co. or Gelled Permethyl® 99A by Penreco may be used.

Examples that may be mentioned include Kraton® G1650 (SEBS), Kraton® G1651 (SEBS), Kraton® G1652 (SEBS), Kraton® G1657X (SEBS), Kraton® G1701X (SEP), Kraton® G1702X (SEP), Kraton® G1726X (SEB), Kraton® D-1101 (SBS), Kraton® D-1102 (SBS), Kraton® D-1107 (SIS), Gelled Permethyl® 99A-750, Gelled Permethyl® 99A-753-58 (blend of triblock and of starburst block polymer), Gelled Permethyl® 99A-753-59 (blend of triblock and of starburst block polymer), Versagel® 5970 and Versagel® 5960 from Penreco (blend of triblock and of starburst polymer in isododecane).

Styrene-methacrylate copolymers may also be used, such as the polymers sold under the references OS 129880, OS 129881, and OS 84383 from Lubrizol (styrene-methacrylate copolymer).

In one embodiment, the at least one film-forming polymer is chosen from copolymers of a vinyl ester (the vinyl group being directly attached to the oxygen atom of the ester group and the vinyl ester having a saturated, linear, or branched hydrocarbon-based radical of 1 to 19 carbon atoms, linked to the carbonyl of the ester group) and of at least one other monomer, which may be a vinyl ester (other than the vinyl ester already present), α-olefins (containing from 8 to 28 carbon atoms), alkyl vinyl ethers (the alkyl group of which contains from 2 to 18 carbon atoms), and allylic or methallylic esters (containing a saturated, linear or branched hydrocarbon-based radical of 1 to 19 carbon atoms, linked to the carbonyl of the ester group).

These copolymers may be partially crosslinked using crosslinking agents, which may be chosen from the vinyl copolymers, allylic copolymers, and methallylic copolymers, such as tetraallyloxyethane, divinylbenzene, divinyl octanedioate, divinyl dodecanedioate, and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Liposoluble film-forming polymers that may also be mentioned include liposoluble copolymers, such as those resulting from the copolymerization of vinyl esters containing from 9 to 22 carbon atoms or of alkyl acrylates or methacrylates, the alkyl radicals containing from 10 to 20 carbon atoms.

Such liposoluble copolymers may be chosen from polyvinyl stearate copolymers, polyvinyl stearate crosslinked with divinylbenzene, with diallyl ether, or with diallyl phthalate, polystearyl (meth)acrylate copolymers, polyvinyl laurate, and polylauryl (meth)acrylate, these poly(meth)acrylates possibly being crosslinked with ethylene glycol dimethacrylate or tetraethylene glycol dimethacrylate.

The liposoluble copolymers defined above are known and described for example in French Patent Application No. FR A 2 232 303. They may have a weight-average molecular weight ranging from 2,000 to 500,000, such as from 4,000 to 200,000.

As examples of liposoluble polymers that may be used, mention may be made of polyalkylenes and C₂-C₂₀ alkene copolymers, for example polybutene.
b) amorphous and liposoluble polycondensates, optionally not comprising any groups donating hydrogen interactions, for example aliphatic polyesters containing C₄₋₅₀ alkyl side chains, polyesters resulting from the condensation of fatty acid dimers, and polyesters comprising a silicone-based segment in the form of a block, graft, or end group, as defined, for example, in French Patent Application No. FR 0 113 920.
c) amorphous and liposoluble polysaccharides comprising alkyl (ether or ester) side chains, such as alkylcelluloses containing a saturated or unsaturated, linear or branched C₁ to C₈ alkyl radical, such as ethylcellulose and propylcellulose.

The at least one film-forming polymer may be chosen from cellulose-based polymers such as nitrocellulose, cellulose acetate, cellulose acetobutyrate, cellulose acetopropionate, ethylcellulose, polyurethanes, acrylic polymers, vinyl polymers, polyvinyl butyrals, alkyd resins, resins derived from aldehyde condensation products, such as arylsulfonamide-formaldehyde resins, for instance toluenesulfonamide-formaldehyde resin, and arylsulfonamide epoxy resins.

Film-forming polymers that may be used include nitrocellulose RS ⅛ sec.; RS ¼ sec.; ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; and SS 5 sec., sold for example by the company Hercules; the toluenesulfonamide-formaldehyde resins Ketjentflex MS80 from the company Akzo, and “Santolite® MHP and Santolite® MS80 from the company Faconnier or Resimpol 80 from the company Pan Americana, the alkyd resin Beckosol® Ode 230-70-E from the company Dainippon, the acrylic resin Acryloid® B66 from the company Rohm & Haas, and the polyurethane resin Trixene® PR 4127 from the company Baxenden.
d) silicone resins, which may be soluble or swellable in silicone oils. These resins are crosslinked polyorganosiloxane polymers.

The nomenclature of silicone resins is known under the name “MDTQ”, the resin being described as a function of the various siloxane monomer units it comprises, each of the letters “MDTQ” characterizing a type of unit.

The letter M represents the monofunctional unit of formula (CH₃)₃SiO_{1/2}, the silicon atom being linked to only one oxygen atom in the polymer comprising this unit.

The letter D denotes a difunctional unit (CH₃)₂SiO_{2/2} in which the silicon atom is linked to two oxygen atoms.

The letter T represents a trifunctional unit of formula (CH₃)SiO_{3/2}.

In the units M, D, and T defined above, at least one of the methyl groups may be substituted with a group R other than a methyl group, such as a hydrocarbon-based radical (for example alkyl) containing from 2 to 10 carbon atoms, a phenyl group, or a hydroxyl group.

Finally, the letter Q means a tetrafunctional unit SiO_{4/2} in which the silicon atom is linked to four hydrogen atoms, which are themselves linked to the polymer residue.

Various resins with different properties may be obtained from these various units, the properties of these polymers varying as a function of the type of monomers (or units), the type and number of substituted radicals, the length of the polymer chain, the degree of branching, and the size of the pendent chains.

Examples of these silicone resins that may be mentioned include:
siloxysilicates, which may be trimethylsiloxysilicates of formula [(CH₃)₃XSiXO]ₓX(SiO_{4/2})_{y} (units MQ) in which x and y are integers ranging from 50 to 80,
polysilsesquioxanes of formula (CH₃SiO_{3/2})ₓ (units T) in which x is greater than 100 and at least one of the methyl radicals of which may be substituted with a group R as defined above,
the polymethylsilsesquioxanes, which are polysilsesquioxanes in which none of the methyl radicals is substituted with another group. Such polymethylsilsesquioxanes are described, for example, in U.S. Pat. No. 5,246,694, the content of which is incorporated by reference herein.

As examples of commercially available polymethylsilsesquioxane resins, mention may be made of those sold:
by the company Wacker under the reference Resin MK, such as Belsil® PMS MK: polymer comprising CH₃SiO_{3/2} repeating units (units T), which may also comprise up to 1% by weight of (CH₃)₂SiO_{2/2} units (units D) and having an average molecular weight of about 10,000,
by the company Shin-Etsu under the references KR-220L, which comprise units T of formula CH₃SiO_{3/2} and Si—OH (silanol) end groups, under the reference KR-242A, which comprise 98% of units T and 2% of dimethyl units D and contain Si—OHOU end groups, and under the reference KR-251, comprising 88% of units T and 12% of dimethyl units D and containing Si—OH end groups.

Siloxysilicate resins that may be mentioned include trimethylsiloxysilicate resins (TMS) optionally in the form of powders. Such resins are sold under the reference SR1000 by the company General Electric and under the reference TMS 803 by the company Wacker. Mention may also be made of trimethylsiloxysilicate resins sold in a solvent such as cyclomethicone, sold under the name KF-7312J by the company Shin-Etsu, and DC 749 and DC 593 by the company Dow Corning.
e) Silicone-based polyorganosiloxane polyamides, such as those described in U.S. Pat. Nos. 5,874,069; 5,919,441; 6,051,216; and 5,981,680.

As disclosed herein, these silicone-based polymers may belong to at least one of the following two families:
1) polyorganosiloxanes comprising at least two groups capable of establishing hydrogen interactions, these two groups being located in the polymer chain; and
2) polyorganosiloxanes comprising at least two groups capable of establishing hydrogen interactions, these two groups being located on grafts or branches.

The polymers comprising two groups capable of establishing hydrogen interactions in the polymer chain may be polymers comprising at least one unit corresponding to the formula: 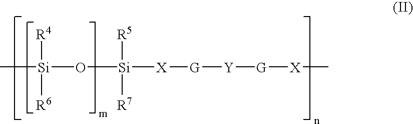
in which:
1) R⁴, R⁵, R⁶ and R⁷, which may be identical or different, each represent a group chosen from:
   linear, branched, or cyclic, saturated or unsaturated, C₁ to C₄₀ hydrocarbon-based groups, optionally comprising in their chain at least one atom chosen from oxygen, sulfur, and nitrogen atoms, and optionally being partially or totally substituted with fluorine atoms,
   C₆ to C₁₀ aryl groups, optionally substituted with at least one C₁ to C₄ alkyl group,
   polyorganosiloxane chains optionally comprising at least one atom chosen from oxygen, sulfur, and nitrogen atoms;
2) the groups X, which may be identical or different, are each chosen from linear or branched C, to C₃₋₀ alkylenediyl groups, optionally comprising in their chains at least one atom chosen from oxygen and nitrogen atoms;
3) Y is chosen from saturated or unsaturated, C₁ to C₅₀ linear or branched divalent alkylene groups, arylene groups, cycloalkylene groups, alkylarylene groups, and arylalkylene groups, optionally comprising at least one atom chosen from oxygen, sulfur, and nitrogen atoms, and/or bearing as substituent at least one of the following atoms or groups of atoms: fluorine, hydroxyl, C₃ to C₈ cycloalkyl, C₁ to C₄₋₀ alkyl, C₅ to C₁₀ aryl, phenyl optionally substituted with 1 to 3 C₁ to C₃ alkyl groups, C₁ to C₃ hydroxyalkyl, and C₁ to C₆ aminoalkyl; or
4) Y is a group corresponding to the formula: 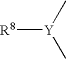
   in which
   T is a linear or branched, saturated or unsaturated, C₃ to C₂₄ trivalent or tetravalent hydrocarbon-based group optionally substituted with a polyorganosiloxane chain, and optionally comprising at least one atom chosen from O, N, and S, or T is a trivalent atom chosen from N, P, and Al, and
   R⁸ is chosen from linear or branched C₁ to C₅₀ alkyl groups and polyorganosiloxane chains, possibly comprising at least one group chosen from ester, amide, urethane, thiocarbamate, urea, thiourea, and sulfonamide groups, which may possibly be linked to another chain of the polymer;
5) the groups G, which may be identical or different, represent divalent groups chosen from: 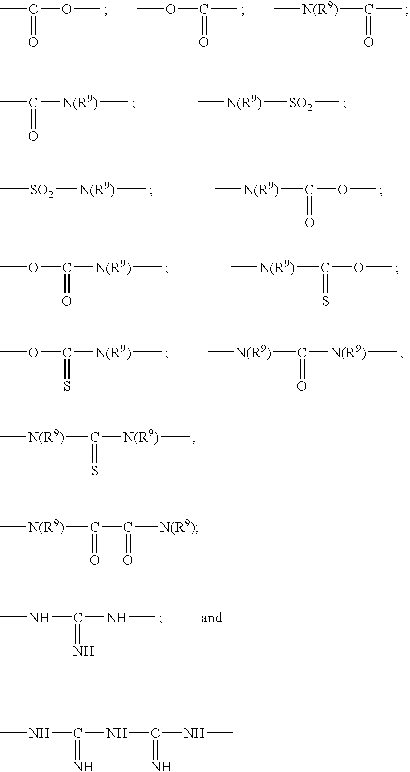
   in which R⁹ is chosen from hydrogen and linear or branched C₁ to C₂₀ alkyl groups, with the proviso that at least 50% of the groups R⁹ of the polymer represent hydrogen and that at least two of the groups G of the polymer are a group other than: 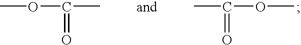
6) n is an integer ranging from 2 to 500, such as from 2 to 200, and m is an integer ranging from 1 to 1,000, such as from 1 to 700 or from 6 to 200.

As disclosed herein, 80% of the groups R⁴, R⁵, R⁶, and R⁷ of the polymer may be chosen from methyl, ethyl, phenyl, and 3,3,3-trifluoropropyl groups.

As disclosed herein, Y can represent various divalent groups, furthermore optionally comprising one or two free valencies to establish bonds with other moieties of the polymer or copolymer. For example, Y may represent a group chosen from:
a) linear C₁ to C₂₀, such as C₁ to C₁₀, alkylene groups,
b) C₃₀ to C₅₆ branched alkylene groups possibly comprising rings and unconjugated unsaturations,
c) C₅-C₆ cycloalkylene groups,
d) phenylene groups optionally substituted with at least one C₁ to C₄₀ alkyl group,
e) C₁ to C₂₀ alkylene groups comprising from 1 to 5 amide groups,
f) C₁ to C₂₀ alkylene groups comprising at least one substituent chosen from hydroxyl, C₃ to C₈ cycloalkane, C₁ to C₃ hydroxyalkyl, and C₁ to C₆ alkylamine groups,
g) polyorganosiloxane chains of formula: 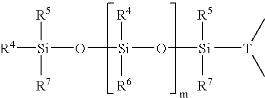
   in which R⁴, R⁵, R⁶, R⁷, T, and m are as defined above, and
h) polyorganosiloxane chains of formula: 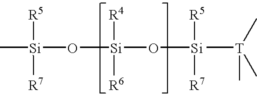
   in which R⁴, R⁵, R⁶, R⁷, and T are as defined above.

The polyorganosiloxanes of the second family may be polymers comprising at least one unit corresponding to formula (III): 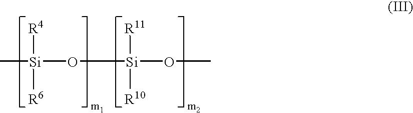
in which
R⁴ and R⁶, which may be identical or different, are as defined above for formula (II),
R¹⁰ is a group as defined above for R⁴ and R⁶, or a group of formula —X-G-R¹² in which X and G are as defined above for formula (II) and R¹² is chosen from hydrogen; linear, branched, or cyclic, saturated or unsaturated, C₁ to C₅₀ hydrocarbon-based groups optionally comprising in its chain at least one atom chosen from O, S, and N, optionally substituted with at least one fluorine atom and at least one hydroxyl group; and phenyl groups optionally substituted with at least one C₁ to C₄ alkyl group,
R¹¹ is a group of formula —X-G-R⁹ in which X, G and R¹² are as defined above,
m₁ is an integer ranging from 1 to 998, and
m₂ is an integer ranging from 2 to 500.

According to certain embodiments, the polymer used may be a homopolymer, that is to say a polymer comprising several identical units, for example units of formula (II) or of formula (III).

According to certain embodiments, it is also possible to use a polymer comprising a copolymer comprising several different units of formula (II), that is to say a polymer in which at least one of the groups R⁴, R⁵, R⁶, R⁷, X, G, Y, m, and n is different in one of the units. The copolymer may also be formed from several units of formula (III), in which at least one of the groups R⁴, R⁶, R¹⁰, R¹¹, m₁, and m₂ is different in at least one of the units.

It is also possible to use a copolymer comprising at least one unit of formula (II) and at least one unit of formula (III), the units of formula (II) and the units of formula (III) possibly being identical to or different from each other.

According to one embodiment, it is also possible to use a copolymer further comprising at least one hydrocarbon-based unit comprising two groups capable of establishing hydrogen interactions, chosen from ester, amide, sulfonamide, carbamate, thiocarbamate, urea, urethane, thiourea, oxamido guanidine, and biguanidino groups.

These copolymers may be chosen from block copolymers and grafted copolymers.

According to the present disclosure, the at least one film-forming polymer may be a solid that is insoluble in the fatty phase of the composition at room temperature, for example at approximately 25° C. The at least one film-forming polymer may also be insoluble in the fatty phase at its softening point, unlike a wax, even of polymeric origin, which is soluble in the liquid organic phase (or fatty phase) at its melting point. In this sense, the at least one film-forming polymer is not a wax.

The composition disclosed herein may comprise at least one stable dispersion of essentially spherical polymer particles of at least one polymer, in a physiologically acceptable fatty phase.

These dispersions may be in the form of polymer nanoparticles in stable dispersion in the liquid organic phase. The nanoparticles may have a mean size ranging from 5 to 800 nm, such as ranging from 50 to 500 nm. However, it is possible to obtain polymer particles ranging up to 1 μm in size.

In certain embodiments, the polymer particles in dispersion are insoluble in water-soluble alcohols, for instance ethanol.

The polymers in dispersion that may be used in the composition disclosed herein may have a molecular weight ranging from 2,000 to 10,000,000 g/mol and a Tg ranging from −100° C. to 300° C., such as ranging from −50° C. to 100° C. or from −10° C. to 50° C.

It is possible to use film-forming polymers having a low Tg, of less than or equal to skin temperature, such as less than or equal to 40° C.

Among the film-forming polymers that may be mentioned are at least one of acrylic or vinyl free-radical homopolymers and copolymers, for example with a Tg of less than or equal to 40° C., such as ranging from −10° C. to 30° C.

As used herein, the term “free-radical polymer” means a polymer obtained by polymerization of unsaturated, for example ethylenic, monomers, each monomer being capable of homopolymerizing (unlike polycondensates). The free-radical polymers may be chosen from vinyl polymers and copolymers, for example acrylic polymers.

The acrylic polymers may result from the polymerization of at least one of ethylenically unsaturated monomers containing at least one acid group, esters of these acid monomers, and amides of these acids.

Monomers bearing an acid group that may be used include α,β-ethylenic unsaturated carboxylic acids such as acrylic acid, methacrylic acid, crotonic acid, maleic acid, and itaconic acid. (Meth)acrylic acid and crotonic acid may be used, and in at least one embodiment, (meth)acrylic acid is used.

The acid monomer esters may be chosen from (meth)acrylic acid esters (also known as (meth)acrylates), for instance alkyl (meth)acrylates, such as of a C₁-C₂₀ or C₁-C₈ alkyl; aryl (meth)acrylates, such as of a C₆-C₁₀ aryl; and hydroxyalkyl (meth)acrylates, such as of a C₂-C₆ hydroxyalkyl. Alkyl (meth)acrylates that may be mentioned include methyl, ethyl, butyl, isobutyl, 2-ethylhexyl, and lauryl (meth)acrylate. Hydroxyalkyl (meth)acrylates that may be mentioned include hydroxyethyl (meth)acrylate and 2-hydroxypropyl (meth)acrylate. Aryl (meth)acrylates that may be mentioned include benzyl acrylate and phenyl acrylate.

The (meth)acrylic acid esters that may be mentioned are the alkyl (meth)acrylates.

Free-radical polymers that may be used include copolymers of (meth)acrylic acid and of alkyl (meth)acrylate, for example of a C₁-C₄ alkyl. Methyl acrylates optionally copolymerized with acrylic acid may be used.

Amides of the acid monomers that may be mentioned include (meth)acrylamides, for example N-alkyl(meth)acrylamides, such as of a C₂-C₁₂ alkyl, such as N-ethylacrylamide, N-t-butylacrylamide, and N-octylacrylamide; and N-di(C₁-C₄)alkyl(meth)acrylamides.

The acrylic polymers may also result from the polymerization of ethylenically unsaturated monomers containing at least one amine group, in free form or in partially or totally neutralized form, or alternatively in partially or totally quaternized form. Such monomers may be, for example, chosen from dimethylaminoethyl (meth)acrylate, dimethylaminoethylmethacrylamide, vinylamine, vinylpyridine, and diallyldimethylammonium chloride.

The vinyl polymers may also result from the homopolymerization or copolymerization of at least one monomer chosen from vinyl esters and styrene monomers. For example, these monomers may be polymerized with at least one of acid monomers, esters thereof, and amides thereof, such as those mentioned above. Examples of vinyl esters that may be mentioned include vinyl acetate, vinyl propionate, vinyl neodecanoate, vinyl pivalate, vinyl benzoate, and vinyl t-butylbenzoate. Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

As other vinyl monomers that may be used, mention may also be made of:
N-vinylpyrrolidone, N-vinylcaprolactam, vinyl-N-(C₁-C₈)alkylpyrroles, vinyloxazoles, vinylthiazoles, vinylpyrimidines, and vinylimidazoles; and
olefins such as ethylene, propylene, butylene, isoprene, and butadiene.

The vinyl polymer may be crosslinked with at least one difunctional monomer, for example comprising at least two ethylenic unsaturations, such as ethylene glycol dimethacrylate and diallyl phthalate.

In a non-limiting manner, the polymers in dispersion as disclosed herein may be chosen from at least one of the following polymers or copolymers: polyurethanes, polyurethane-acrylics, polyureas, polyurea-polyurethanes, polyester-polyurethanes, polyether-polyurethanes, polyesters, polyesteramides, alkyds, acrylic polymers, acrylic copolymers, vinyl polymers, vinyl copolymers, acrylic-silicone copolymers, polyacrylamides, silicone polymers, for instance silicone polyurethanes and silicone acrylics, and fluoro polymers.

The polymers in dispersion in the fatty phase may be present in an amount ranging from 5% to 40%, such as from 5% to 35% or from 8% to 30%, by weight relative to the total weight of solids in the composition.

According to one embodiment, the polymer particles in dispersion are surface-stabilized with at least one stabilizer that is solid at room temperature. In this case, the amount of solids in the dispersion represents the total amount of polymer plus stabilizer, given that the amount of polymer cannot be less than 5%.

The polymer particles may be surface-stabilized by means of at least one stabilizer that may be chosen from block polymers, grafted polymers, and random polymers. The stabilization may take place by any known means, for example by direct addition of the stabilizing polymer during the polymerization.

The at least one stabilizer may also be present in the mixture before polymerization of the polymer. However, it is also possible to add it continuously, for example when the monomers are also added continuously.

2-30% by weight, such as 5-20% by weight, of stabilizer may be used relative to the weight of the initial monomer mixture.

When a grafted polymer and/or a block polymer is used as stabilizer, the synthesis solvent is chosen such that at least some of the grafts or blocks of the polymer-stabilizer are soluble in the solvent, the rest of the grafts or blocks being insoluble therein. The polymer-stabilizer used during the polymerization should be soluble or dispersible in the synthesis solvent. Furthermore, a stabilizer whose insoluble blocks or grafts have a certain affinity for the polymer formed during the polymerization may be chosen.

Among the grafted polymers that may be mentioned are silicone polymers grafted with a hydrocarbon-based chain and hydrocarbon-based polymers grafted with a silicone chain.

Thus, grafted-block or block copolymers comprising at least one polyorganosiloxane block and at least one block of a free-radical polymer, for instance grafted acrylic/silicone copolymers, may thus be used, which may be used when the non-aqueous medium contains silicone.

It is also possible to use grafted-block or block copolymers comprising at least one polyorganosiloxane block and at least one block of a polyether. The polyorganopolysiloxane block may be a polydimethylsiloxane or a poly(C₂-C₁₈)alkylmethylsiloxane. The polyether block may be a poly(C₂-C₁₈)alkylene, such as at least one of polyoxyethylene and polyoxypropylene. For example, dimethicone copolyols and (C₂-C₁₈)alkyldimethicone copolyols such as those sold under the name Dow Corning 3225C by the company Dow Corning, and lauryl methicones such as those sold under the name Dow Corning Q2-5200 by the company Dow Corning, may be used.

Grafted-block or block copolymers that may also be mentioned include those comprising at least one block resulting from the polymerization of at least one ethylenic monomer containing at least one optionally conjugated ethylenic bond, for instance ethylene and dienes such as butadiene and isoprene, and of at least one block of a vinyl polymer, or a styrene polymer. When the ethylenic monomer comprises several optionally conjugated ethylenic bonds, the residual ethylenic unsaturations after the polymerization may hydrogenated. Thus, in a known manner, the polymerization of isoprene leads, after hydrogenation, to the formation of an ethylene-propylene block, and the polymerization of butadiene leads, after hydrogenation, to the formation of an ethylene-butylene block. Among these polymers that may be mentioned are block copolymers, for example diblock copolymers and triblock copolymers, such as polystyrene/polyisoprene (SI), polystyrene/polybutadiene (SB) such as those sold under the name Luvitole HSB by BASF, of the type such as polystyrene/copoly(ethylene-propylene) (SEP) such as those sold under the name Kraton® by Shell Chemical Co. and of the type such as polystyrene/copoly(ethylene-butylene) (SEB). Kraton® G1650 (SEBS), Kraton® G1651 (SEBS), Kraton® G1652 (SEBS), Kraton® G1657X (SEBS), Kraton® G1701X (SEP), Kraton® G1702X (SEP), Kraton® G1726X (SEB), Kraton® D-1101 (SBS), Kraton® D-1102 (SBS), and Kraton® D-1107 (SIS) may be used. The polymers are generally known as hydrogenated or non-hydrogenated diene copolymers.

Gelled Permethyl® 99A-750, 99A-753-59 and 99A-753-58 (mixture of triblock and of star polymer), Versagel® 5960 from Penreco (triblock+star polymer); OS129880, OS129881, and OS84383 from Lubrizol (styrene/methacrylate copolymer) may also be used.

As grafted-block or block copolymers comprising at least one block resulting from the polymerization of at least one ethylenic monomer comprising at least one ethylenic bond and of at least one block of an acrylic polymer, mention may be made of poly(methyl methacrylate)/polyisobutylene diblock copolymers, poly(methyl methacrylate)/polyisobutylene triblock copolymers, and grafted copolymers containing a poly(methyl methacrylate) skeleton and polyisobutylene grafts.

As grafted-block or block copolymers comprising at least one block resulting from the polymerization of at least one ethylenic monomer comprising at least one ethylenic bond and of at least one block of a polyether such as a C₂-C₁₈ polyalkylene (for example polyethylene and polyoxypropylene), mention may be made of polyoxyethylene/polybutadiene diblock copolymers, polyoxyethylene/polybutadiene triblock copolymers, polyoxyethylene/polyisobutylene diblock copolymers, and polyoxyethylene/polyisobutylene triblock copolymers.

When a random polymer is used as stabilizer, it is chosen such that it has a sufficient amount of groups making it soluble in the intended synthesis solvent.

Copolymers based on alkyl acrylates or methacrylates derived from C₁-C₄ alcohols and on alkyl acrylates or methacrylates derived from C₈-C₃₀ alcohols may thus be used. Mention may be made of stearyl methacrylate/methyl methacrylate copolymer.

When the synthesis solvent of the polymer is apolar, one may choose as stabilizer a polymer that provides the fullest possible coverage of the particles, several polymer-stabilizer chains then being absorbed onto a particle of polymer obtained by polymerization.

In this case, one may use as stabilizer either a grafted polymer or a block polymer, so as to have better interfacial activity. For example, blocks or grafts that are insoluble in the synthesis solvent may provide bulkier coverage at the surface of the particles.

When the synthesis solvent comprises at least one silicone oil, the at least one stabilizer may be chosen from grafted-block copolymers and block copolymers comprising at least one polyorganosiloxane block and at least one block chosen from free-radical polymers, polyethers, and polyesters, for instance polyoxypropylene and oxyethylene blocks.

When the synthesis solvent does not comprise any silicone oil, the at least one stabilizer may be chosen from:
(a) grafted-block or block copolymers comprising at least one polyorganosiloxane block and at least one block chosen from free-radical polymers, polyethers, and polyesters;
(b) copolymers of alkyl acrylates or methacrylates derived from C₁-C₄ alcohols and of alkyl acrylates or methacrylates derived from C₈-C₃₀ alcohols; and
(c) grafted-block or block copolymers comprising at least one block resulting from the polymerization of at least one ethylenic monomer containing conjugated ethylenic bonds, and at least one block chosen from vinyl polymers, acrylic polymers, polyethers, and polyesters.

Diblock polymers may be used as stabilizer.

The at least one film-forming polymer that is liposoluble or in dispersion in a fatty phase may also be used in an amount ranging from 0.01% to 20% (as active material), for instance from 1% to 10%, where appropriate, relative to the total weight of the composition.

According to another embodiment, the at least one film-forming polymer may be chosen from aqueous dispersions of polymer particles, in the case where the composition disclosed herein comprises an aqueous phase.

The aqueous dispersion comprising at least one film-forming polymer may be prepared by a person skilled in the art on the basis of his general knowledge, such as by emulsion polymerization or by dispersion of the preformed polymer.

Among the film-forming polymers which may be used in the composition disclosed herein, mention may be made of synthetic polymers, polycondensate polymers, free-radical polymers, polymers of natural origin, and mixtures thereof.

Among the polycondensates, mention may also be made of anionic polyurethanes, cationic polyurethanes, nonionic polyurethanes, amphoteric polyurethanes, polyurethane-acrylics, polyurethane-polyvinylpyrrolidones, polyester-polyurethanes, polyether-polyurethanes, polyureas, polyurea/polyurethanes, and mixtures thereof.

The polyurethanes may be, for example, chosen from aliphatic, cycloaliphatic or aromatic polyurethanes, polyurea/polyurethanes, and polyurea copolymers, containing:
at least one block of linear or branched, aliphatic, cycloaliphatic and/or aromatic polyester origin,
at least one block of aliphatic, cycloaliphatic and/or aromatic polyether origin,
at least one substituted or unsubstituted, branched or unbranched silicone block, for example polydimethylsiloxane and polymethylphenylsiloxane, and/or
at least one block comprising fluoro groups.

The polyurethanes as defined herein may also be obtained from branched or unbranched polyesters or from alkyds containing mobile hydrogens, which are modified by means of a polyaddition with a diisocyanate and a difunctional organic co-reactive compound (for example dihydro, diamino, and hydroxyamino), and may also comprise at least one group chosen from carboxylic acid groups, carboxylate groups, sulfonic acid groups, sulfonate groups, neutralizable tertiary amine groups, and quaternary ammonium groups.

Mention may also be made of polyesters, polyesteramides, fatty-chain polyesters, polyamides, and epoxyester resins.

The polyesters may be obtained, in a known manner, by polycondensation of aliphatic or aromatic diacids with aliphatic or aromatic diols or with polyols. Aliphatic diacids may be chosen from at least one of succinic acid, glutaric acid, adipic acid, pimelic acid, suberic acid, and sebacic acid. Aromatic diacids may be chosen from at least one of terephthalic acid, isophthalic acid, and derivatives such as phthalic anhydride. Aliphatic diols may be chosen from at least one of ethylene glycol, propylene glycol, diethylene glycol, neopentyl glycol, cyclohexanedimethanol, and 4,4-N-(1-methylpropylidene)bisphenol. Polyols may be chosen from at least one of glycerol, pentaerythritol, sorbitol, and trimethylolpropane.

The polyesteramides may be obtained in a similar manner to the polyesters, by polycondensation of diacids with diamines or amino alcohols. Diamines may be chosen from at least one of ethylenediamine, hexamethylenediamine, or meta-phenylenediamine, and para-phenylenediamine. Monoethanolamine may be used as amino alcohol.

As monomer bearing an anionic group which may be used during the polycondensation, mention may be made, for example, of dimethylolpropionic acid, trimellitic acid, derivatives such as trimellitic anhydride, the sodium salt of pentanediol-3-sulfonic acid, and the sodium salt of 5-sulfo-1,3-benzenedicarboxylic acid. The fatty-chain polyesters may be obtained using fatty-chain diols during the polycondensation. The epoxy ester resins may be obtained by polycondensation of fatty acids with a condensate having α,ω-diepoxy ends.

The free-radical polymers may be chosen from acrylic polymers, acrylic copolymers, vinyl polymers, and vinyl copolymers. Anionic radical polymers may be mentioned. As examples of monomers bearing an anionic group which may be used during the free-radical polymerization, mention may be made of acrylic acid, methacrylic acid, crotonic acid, maleic anhydride, and 2-acrylamido-2-methylpropanesulfonic acid.

The acrylic polymers may result from the copolymerization of monomers chosen from the acrylic acid esters, acrylic acid amides, methacrylic acid esters, and methacrylic acid amides. As examples of ester monomers, mention may be made of methyl methacrylate, ethyl methacrylate, butyl methacrylate, isobutyl methacrylate, 2-ethylhexyl methacrylate, and lauryl methacrylate. As examples of amide monomers, mention may be made of N-t-butylacrylamide and N-t-octylacrylamide.

Acrylic polymers obtained by copolymerization of ethylenically unsaturated monomers containing hydrophilic groups, for example of nonionic nature, such as hydroxyethyl acrylate, 2-hydroxypropyl acrylate, hydroxyethyl methacrylate, and 2-hydroxypropyl methacrylate, may be used.

The vinyl polymers may result from the homopolymerization or copolymerization of monomers chosen from vinyl esters, styrene, and butadiene. As examples of vinyl esters, mention may be made of vinyl acetate, vinyl neodecanoate, vinyl pivalate, vinyl benzoate, and vinyl t-butylbenzoate.

Acrylic/silicone copolymers or nitrocellulose/acrylic copolymers may also be used.

Mention may also be made of the polymers resulting from the free-radical polymerization of at least one free-radical monomer inside and/or partially at the surface of preexisting particles of at least one polymer chosen from polyurethanes, polyureas, polyesters, polyesteramides, and alkyds. These polymers may be referred to as “hybrid polymers”.

When an aqueous dispersion of polymer particles is used, the solids content of the aqueous dispersion may range from about 3% to 60%, such as from 10% to 50%, by weight.

The size of the polymer particles in aqueous dispersion may range from 10 to 500 nm, such as from 20 to 150 nm, which may allow the production of a film of noteworthy gloss. However, particle sizes ranging up to 1 micron may be used.

Aqueous dispersions of film-forming polymers that may be used include: the acrylic dispersions sold under the names Neocryl® XK-90, Neocryl® A-1070, Neocryl® A-1090, Neocryl® BT-62, Neocryl® A-1079, and Neocryl® A-523 by the company Avecia-Neoresins; Dow Latex® 432 by the company Dow Chemical; Daitosol 5000 AD and Daitosol 5000 SJ by the company Daito Kasey Kogyo; Syntran® 5760 by the company Interpolymer; the aqueous dispersions of polyurethane sold under the names Neorez® R-981 and Neorez® R-974 by the company Avecia-Neoresins; Avalure® UR-405, Avalure® UR-410, Avalure® UR-425, Avalure® UR-450, Sancure® 875, Sancure® 861, Sancure® 878, and Sancure® 2060 by the company Goodrich; Impranil® 85 by the company Bayer; Aquamere® H-1511® by the company Hydromer; the sulfopolyesters sold under the brand name Eastman® AQ by the company Eastman Chemical Products; vinyl dispersions, for instance Mexomer PAM, aqueous dispersions of polyvinyl acetate, for instance Vinybran from the company Nisshin Chemical and those sold by the company Union Carbide; aqueous dispersions of terpolymer of vinylpyrrolidone, dimethylaminopropylmethacrylamide, and lauryldimethylpropylmethacrylamidoammonium chloride, such as Styleze® W from ISP; aqueous dispersions of polyurethane/polyacrylic hybrid polymers, such as those sold under the references Hybridur® by the company Air Products and Duromer® from National Starch, dispersions of core/shell type: for example those sold by the company Atofina under the reference Kynar® (core:fluoro-shell:acrylic) those described in U.S. Pat. No. 5,188,899 (core:silica-shell:silicone), and mixtures thereof.

In the case where the composition comprises an aqueous phase, the at least one film-forming polymer may be a water-soluble polymer. The water-soluble polymer is thus dissolved in the aqueous phase of the composition.

Among the water-soluble film-forming polymers that may be mentioned are the following cationic polymers:
(1) acrylic polymers and acrylic copolymers, such as polyacrylates and polymethacrylates; the copolymers of the family (1) may also comprise at least one unit derived from comonomers that may be chosen from the family of acrylamides, methacrylamides, diacetoneacrylamides, acrylamides, and methacrylamides substituted on the nitrogen with lower alkyls, acrylic acids, methacrylic acids, or esters thereof; vinyllactams such as vinylpyrrolidone and vinylcaprolactam; and vinyl esters.

Thus, among these copolymers of the family (1), mention may be made of:
copolymers of acrylamide and of dimethylaminoethyl methacrylate, quaternized with an entity chosen from dimethyl sulfate and dimethyl halides, such as the product sold under the name Hercofloc® by the company Hercules,
the copolymer of acrylamide and of methacryloyloxyethyltrimethylammonium chloride described, for example, in European Patent Application No. EP A 080 976 and sold under the name Bina Quat P 100 by the company Ciba Geigy,
the copolymer of acrylamide and of methacryloyloxyethyltrimethylammonium methosulfate sold under the name Reten® by the company Hercules,
quaternized or non-quaternized copolymers of vinylpyrrolidone/dialkylaminoalkyl acrylate and of vinylpyrrolidone/dialkylaminoalkyl methacrylate, such as the products sold under the name Gafquat® by the company ISP, for instance Gafquat® 734 and Gafquat® 755, and the products denoted as Copolymer 845, 958, and 937. These polymers are described, for example, in detail in French Patent Nos. 2 077 143 and 2 393 573,
terpolymers of dimethylaminoethyl methacrylate/vinylcaprolactam/vinylpyrrolidone, such as the product sold under the name Gaffix® VC 713 by the company ISP, and
the quaternized copolymer of vinylpyrrolidone/dimethylaminopropylmethacrylamide, such as the product sold under the name Gafquat® HS 100 by the company ISP.
   (2) the quaternized polysaccharides described more for example in U.S. Pat. Nos. 3,589,578 and 4,031,307, such as guar gums containing trialkylammonium cationic groups. Such products are sold for example under the trade names Jaguar® C13S, Jaguar® C15, and Jaguar® C17 by the company Meyhall.
   (3) quaternary copolymers of vinylpyrrolidone and of vinylimidazole;
   (4) chitosans and salts thereof;
   (5) cationic cellulose derivatives such as copolymers of cellulose and of cellulose derivatives grafted with a water-soluble monomer comprising a quaternary ammonium, and described for example in U.S. Pat. No. 4,131,576, such as hydroalkylcelluloses, for instance hydroxymethyl-, hydroxyethyl-, and hydroxypropylcelluloses grafted for example with a salt chosen from methacryloyloxyethyltrimethylammonium, methacrylamidopropyltrimethylammonium, and dimethyldiallylammonium salts. The products sold corresponding to this definition are, for example, the products sold under the name Celquat® L 200 and Celquat® H 100 by the company National Starch.

Among the at least one film-forming water-soluble polymer that may be mentioned are the following amphoteric polymers:
(1) polymers resulting from the copolymerization of a monomer derived from a vinyl compound bearing a carboxylic group such as, for example, acrylic acid, methacrylic acid, maleic acid, α-chloroacrylic acid, and a basic monomer derived from a substituted vinyl compound containing at least one basic atom, such as, for example, a dialkylaminoalkyl methacrylate and acrylate, and a dialkylaminoalkylmethacrylamide and -acrylamide. Such compounds are described for example in U.S. Pat. No. 3,836,537.
(2) Polymers comprising units derived from:
a) at least one monomer chosen from acrylamides and methacrylamides substituted on the nitrogen with an alkyl radical,
b) at least one acidic comonomer comprising at least one reactive carboxylic group, and
c) at least one basic comonomer such as esters containing primary, secondary, tertiary, and quaternary amine substituents of acrylic and methacrylic acids and the product of quaternization of dimethylaminoethyl methacrylate with dimethyl or diethyl sulfate.
(3) crosslinked alkylpolyaminoamides totally or partially derived from polyaminoamides.
(4) polymers comprising zwitterionic units.
(5) chitosan-based polymers.
(6) polymers derived from the N-carboxyalkylation of chitosan, such as N-carboxymethylchitosan and N-carboxybutylchitosan sold under the name Evalsan by the company Jan Dekker.
(7) (C₁-C₅)alkyl vinyl ether/maleic anhydride copolymers, partially modified by a semi-amidation with an N,N-dialkylaminoalkylamine, such as N,N-dimethylaminopropylamine or by a semi-esterification with an N,N-dialkanolamine. These copolymers may also comprise other vinyl comonomers, such as vinylcaprolactam.

The at least one water-soluble film-forming polymer may be chosen from:
proteins, for instance proteins of plant origin such as wheat proteins and soybean proteins; proteins of animal origin such as keratin, for example keratin hydrolysates and sulfonic keratins;
anionic, cationic, amphoteric, or nonionic chitin and chitosan polymers;
polymers of cellulose such as hydroxyethylcellulose, hydroxypropylcellulose, methylcellulose, ethylhydroxyethylcellulose, and carboxymethylcellulose, and quaternized cellulose derivatives;
acrylic polymers and acrylic copolymers, such as polyacrylates and polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate;
copolymers of vinylpyrrolidone and of caprolactam; polyvinyl alcohols;
polymers of natural origin, which are optionally modified, such as at least one of:
gum arabic, guar gum, xanthan derivatives, karaya gum;
alginates and carrageenans;
glycosaminoglycans, hyaluronic acid, and derivatives thereof;
shellac resin, sandarac gum, dammar resins, elemi gums, and copal resins;
deoxyribonucleic acid;
mucopolysaccharides such as hyaluronic acid and chondroitin sulfate, and mixtures thereof.

These polymers may be used, for example, if a more or less appreciable removal of the film by water is desired.

In order to improve the film-forming nature of an oily or aqueous polymer, it is possible to add to the polymer system a coalescer, which may be chosen from known coalescers.

According to one embodiment, the at least one film-forming polymer may be chosen from polymers with a non-silicone organic skeleton grafted with monomers containing a polysiloxane. These polymers may be liposoluble, lipodispersible, water-soluble or dispersible in aqueous medium, where appropriate.

The polymers containing a non-silicone organic skeleton grafted with monomers containing a polysiloxane comprising an organic main chain formed from organic monomers not comprising silicone, onto which is grafted, within the chain and also optionally on at least one of its ends, at least one polysiloxane macromer.

In the text hereinbelow, in accordance with what is generally accepted, the expression “polysiloxane macromer” is understood to refer to any monomer containing a polysiloxane-type polymer chain in its structure.

The non-silicone organic monomers comprising the main chain of the grafted silicone polymer can be chosen from free-radical-polymerizable monomers containing ethylenic unsaturation, polycondensation-polymerizable monomers, such as those forming polyamides, polyesters, and polyurethanes, and ring-opening monomers, such as oxazoline ring-opening monomers and caprolactone ring-opening monomers.

The polymers containing a non-silicone organic skeleton grafted with monomers containing a polysiloxane, in accordance with the present disclosure, can be obtained according to any means known to those skilled in the art, for example by reaction between (i) a starting polysiloxane macromer which is correctly functionalized on the polysiloxane chain and (ii) at least one non-silicone organic compound, itself correctly functionalized with a function which is capable of reacting with at least one functional group borne by the silicone, forming a covalent bond. One example of such a reaction is the free-radical reaction between a vinyl group borne on one of the ends of the silicone with a double bond of a monomer containing ethylenic unsaturation in the main chain.

The polymers containing a non-silicone organic skeleton grafted with monomers containing a polysiloxane, in accordance with the present disclosure, may be chosen from those described in U.S. Pat. Nos. 4,693,935, 4,728,571, and 4,972,037; European Patent Application Nos. EP A 0 412 704, EP A 0 412 707, and EP-A-0 640 105, and PCT Patent Application No. WO 95/00578. These are copolymers obtained by free-radical polymerization starting with monomers containing ethylenic unsaturation and monomers having a terminal vinyl group, or alternatively copolymers obtained by reaction of a polyolefin comprising functionalized groups and a polysiloxane macromer having a terminal function which is reactive with the functionalized groups.

One family of grafted silicone polymers that is suitable for carrying out certain embodiments disclosed herein comprises grafted silicone polymers comprising:
a) from 0 to 98% by weight of at least one free-radical-polymerizable lipophilic monomer (A) of low lipophilic polarity containing ethylenic unsaturation;
b) from 0 to 98% by weight of at least one polar hydrophilic monomer (B) containing ethylenic unsaturation, which is copolymerizable with at least one monomer of the type (A);
c) from 0.01% to 50% by weight of at least one polysiloxane macromer (C) of general formula:
   X(Y)ₙSi(R)₃₋ₘZₘ (I)
   in which:
   X is a vinyl group which is copolymerizable with the monomers (A) and (B);
   Y is a divalent bonding group;
   R is chosen from hydrogen, C₁-C₆ alkyl radicals, C₁-C₆ alkoxy radicals, and C₆-C₁₂ aryl radicals;
   Z is a monovalent polysiloxane unit with a number-average molecular weight of at least 500;
   n is 0 or 1 and m is an integer ranging from 1 to 3; the percentages being calculated relative to the total weight of the monomers (A), (B), and (C).

These polymers have a number-average molecular weight ranging from 10,000 to 2,000,000, and for example may have a glass transition temperature Tg or a crystal melting temperature Tm of at least −20° C.

As examples of lipophilic monomers (A), mention may be made of acrylic or methacrylic acid esters of C₁-C₁₈ alcohols; methacrylic acid esters of C₁₂-C₃₀ alcohols; styrene; polystyrene macromers; vinyl acetate; vinyl propionate; α-methylstyrene; tert-butylstyrene; butadiene; cyclohexadiene; ethylene; propylene; vinyltoluene; acrylic acid esters of 1,1-dihydroperfluoroalkanols and of homologues thereof; methacrylic acid esters of 1,1-dihydroperfluoroalkanols and of homologues thereof; acrylic acid esters of ω-hydrofluoroalkanols, methacrylic acid esters of ω-hydrofluoroalkanols; acrylic acid esters of fluoroalkylsulfonamido alcohols, methacrylic acid esters of fluoroalkylsulfonamido alcohols; acrylic acid esters of fluoroalkyl alcohols, methacrylic acid esters of fluoroalkyl alcohols; acrylic acid esters of fluoroether alcohols, and methacrylic acid esters of fluoroether alcohols, and mixtures thereof. The monomers (A) may be chosen from n-butyl methacrylate, isobutyl methacrylate, tert-butyl acrylate, tert-butyl methacrylate, 2-ethylhexyl methacrylate, methyl methacrylate, 2-(N-methylperfluorooctanesulfonamido)ethyl acrylate, and 2-(N-butylperfluorooctanesulfonamido)ethyl acrylate, and mixtures thereof.

As examples of polar monomers (B), mention may be made of acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and hemiesters thereof, hydroxyalkyl (meth)acrylates, diallyldimethylammonium chloride, vinylpyrrolidone, vinyl ethers, maleimides, vinylpyridine, vinylimidazole, heterocyclic vinyl polar compounds, styrene sulfonate, allyl alcohol, vinyl alcohol, and vinylcaprolactam, and mixtures thereof. The monomers (B) may, for example, be chosen from acrylic acid, N,N-dimethylacrylamide, dimethylaminoethyl methacrylate, quaternized dimethylaminoethyl methacrylate, and vinylpyrrolidone, and mixtures thereof.

Mention is made of the product KP 561 or KP 562 sold by Shin-Etsu such that the monomer (A) is chosen from esters of a C₁₈-C₂₂ alcohol and of methacrylic acid.

The polysiloxane macromers (C) of formula (I) may be chosen from those corresponding to the general formula (II) below: 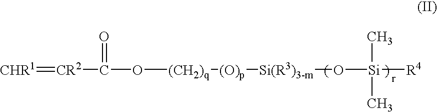
in which:
R¹ is chosen from hydrogen and —COOH (for example, hydrogen);
R² is chosen from hydrogen, methyl, and —CH₂COOH (for example, methyl);
R³ is chosen from C₁-C₆ alkyl, C₁-C₆ alkyl alkoxy, C₁-C₆ alkyl alkylamino, C₆-C₁₂ aryl, and hydroxyl (for example, methyl);
R⁴ is chosen from C₁-C₆ alkyl, C₁-C₆ alkoxy, C₁-C₆ alkylamino, C₆-C₁₂ aryl, and hydroxyl (for example, methyl);
q is an integer ranging from 2 to 6 (for example, 3);
p is 0 or 1;
r is an integer ranging from 5 to 700;
m is an integer ranging from 1 to 3 (for example, 1).

The polysiloxane macromers of formula: 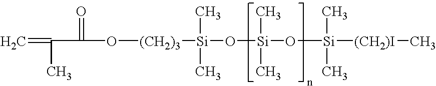
with n being a number ranging from 5 to 700 and I being an integer between 0 and 3, may be used.

One embodiment disclosed herein comprises using a copolymer which may be obtained by free-radical polymerization starting with the monomer mixture comprising:
a) 60% by weight of tert-butyl acrylate;
b) 20% by weight of acrylic acid;
c) 20% by weight of silicone macromer of formula: 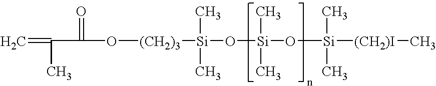
   with n being a number ranging from 5 to 700 and I being an integer between 0 and 3; the weight percentages being calculated relative to the total weight of the monomers.

Another embodiment disclosed herein comprises using a copolymer which may be obtained by free-radical polymerization starting with the monomer mixture comprising:
a) 80% by weight of tert-butyl acrylate;
b) 20% by weight of silicone macromer of formula: 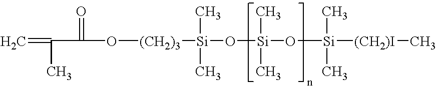
   with n being a number ranging from 5 to 700 and I being an integer from 0 to 3; the weight percentages being calculated relative to the total weight of the monomers.

Another family of grafted silicone polymers with a non-silicone organic skeleton that is suitable for carrying out certain embodiments disclosed herein comprises grafted silicone copolymers which may be obtained by reactive extrusion-molding of a polysiloxane macromer with a reactive terminal function on a polyolefin polymer comprising reactive groups capable of reacting with the terminal function of the polysiloxane macromer to form a covalent bond for grafting the silicone onto the main chain of the polyolefin. These polymers are described, along with a process for their preparation, in Patent Application No. WO 95/00578.

The reactive polyolefins may be chosen from polyethylenes and polymers of ethylene-derived monomers such as propylene, styrene, alkylstyrene, butylene, butadiene, (meth)acrylates, vinyl esters or equivalents, comprising reactive functions capable of reacting with the terminal function of the polysiloxane macromer. They may be chosen from copolymers of ethylene, of ethylene derivatives, and of monomers chosen from those comprising a carboxylic function such as (meth)acrylic acid; those comprising an acid anhydride function such as maleic anhydride; those comprising an acid chloride function such as (meth)acryloyl chloride; those comprising an ester function such as (meth)acrylic acid esters; and those comprising an isocyanate function.

The silicone macromers may be chosen from polysiloxanes comprising a functionalized group, at the end of the polysiloxane chain or close to the end of the chain, chosen from alcohols, thiols, epoxy groups, primary and secondary amines, and from those corresponding to the general formula:
T-(CH₂)₆—Si—[—(OSiR⁵R⁶)ₜ—R⁷]_{y} (III)
in which
T is chosen from NH₂, NHRN, epoxy radicals, OH, and SH radicals;
R⁵, R⁶, R⁷, and RN independently may be chosen from C₁-C₆ alkyl radicals, phenyl radicals, benzyl radicals, C₆-C₁₂ alkylphenyl radicals, and hydrogen;
s is a number ranging from 2 to 100;
t is a number ranging from 0 to 1,000, and
y is a number ranging from 1 to 3.
   They have a number-average molecular weight that may range from 5,000 to 300,000, such as from 8,000 to 200,000 or from 9,000 to 40,000.

According to one embodiment, the at least one film-forming polymer may be purchased from the Minnesota Mining and Manufacturing Company under the trade name Silicone Plus polymers. For example, poly(isobutyl methacrylate-co-methyl FOSEA)-g-poly(dimethylsiloxane) is sold under the trade name SA 70-5 IBMMF.

According to another embodiment, the at least one film-forming polymer is chosen from silicone polymers grafted with non-silicone organic monomers. These polymers may be chosen from liposoluble, lipodispersible, water-soluble polymers, and polymers dispersible in aqueous medium, where appropriate.

The at least one grafted silicone polymer containing a polysiloxane skeleton grafted with non-silicone organic monomers comprising a silicone (or polysiloxane (/SiO—)ₙ) main chain onto which is grafted, within the chain and also optionally on at least one of its ends, at least one organic group not comprising silicone.

The polymers containing a polysiloxane skeleton grafted with non-silicone organic monomers, as disclosed herein, can be existing commercial products or alternatively can be obtained by any means known to those skilled in the art, for example by reaction between (i) a starting silicone which is correctly functionalized on at least one of these silicon atoms, and (ii) a non-silicone organic compound which is itself correctly functionalized with a function which is capable of reacting with at least one functional group borne by the silicone, forming a covalent bond. One example of such a reaction is the hydrosilylation reaction between /Si—H groups and vinyl groups CH₂═CH—, or alternatively the reaction between thio functional groups —SH with these same vinyl groups.

Examples of polymers containing a polysiloxane skeleton grafted with non-silicone organic monomers that are suitable for carrying out certain embodiments disclosed herein, and also their specific mode of preparation, are described for example in European Patent Application No. EP A 0 582 152 and PCT Patent Application No. WO 93/23009 and WO 95/03776, the teachings of which are incorporated by reference herein.

According to one embodiment disclosed herein, the silicone polymer containing a polysiloxane skeleton grafted with non-silicone organic monomers which is used, comprises the result of a free-radical copolymerization between, on the one hand, at least one non-silicone anionic organic monomer containing ethylenic unsaturation and/or a non-silicone hydrophobic organic monomer containing ethylenic unsaturation, and, on the other hand, a silicone containing in its chain at least one, and optionally several, functional groups capable of reacting with the ethylenic unsaturations of the non-silicone monomers, forming a covalent bond, for example thio functional groups.

According to the present disclosure, the anionic monomers containing ethylenic unsaturation may be chosen from at least one linear or branched, unsaturated carboxylic acids, optionally partially or totally neutralized in the form of a salt, it being possible for the at least one unsaturated carboxylic acids to be, for example, chosen from acrylic acid, methacrylic acid, maleic acid, itaconic acid, fumaric acid, and crotonic acid. The suitable salts are, for example, alkali metal salts, alkaline-earth metal salts, and ammonium salts. It will likewise be noted that, in the final grafted silicone polymer, the organic group of anionic nature which comprises the result of the free-radical (homo)polymerization of at least one anionic monomer of unsaturated carboxylic acid type can, after reaction, be post-neutralized with a base (sodium hydroxide, aqueous ammonia, etc.) in order to place it in the form of a salt.

According to the present disclosure, the hydrophobic monomers containing ethylenic unsaturation may be chosen from at least one of acrylic acid esters of alkanols and methacrylic acid esters of alkanols. The alkanols may be of C₁-C₃₀, for example of C₁-C₂₂. The monomers may be chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, and stearyl (meth)acrylate, and mixtures thereof.

One family of silicone polymers containing a polysiloxane skeleton grafted with non-silicone organic monomers that may be suitable for carrying out certain embodiments disclosed herein may comprise silicone polymers comprising in their structure the unit of formula IV below: 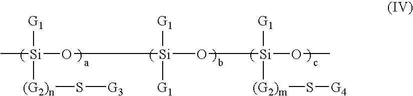
in which
the radicals G₁, which may be identical or different, are chosen from hydrogen, C₁-C₁₀ alkyl radicals, and phenyl radicals;
the radicals G₂, which may be identical or different, are chosen from C₁-C₁₀ alkylene groups;
G₃ is a polymer residue resulting from the (homo)polymerization of at least one anionic monomer containing ethylenic unsaturation;
G₄ is a polymer residue resulting from the (homo)polymerization of at least one hydrophobic monomer containing ethylenic unsaturation;
m and n are equal to 0 or 1;
a is an integer ranging from 0 to 50;
b is an integer ranging from 10 to 350;
c is an integer ranging from 0 to 50;
with the proviso that one of the parameters a and c is other than 0.

In certain embodiments, the unit of formula (IV) above has at least one, and for example all, of the following characteristics:
the radicals G₁ denote an alkyl radical, such as a methyl radical;
n is not zero, and the radicals G₂ represent a divalent C₁-C₃ radical, such as a propylene radical;
G₃ represents a polymer radical resulting from the (homo)polymerization of at least one monomer of the carboxylic acid type containing ethylenic unsaturation, for example at least one of acrylic acid and methacrylic acid;
G₄ represents a polymer radical resulting from the (homo)polymerization of at least one monomer of the (C₁-C₁₀) alkyl (meth)acrylate type, for example at least one of isobutyl and methyl (meth)acrylate.

Examples of silicone polymers corresponding to formula (IV) are, for example polydimethylsiloxanes (PDMSs) onto which are grafted, via a thiopropylene-type secondary bond, mixed poly(meth)acrylic acid polymer units and polyalkyl (meth)acrylate polymer units.

Other examples of silicone polymers corresponding to formula (IV) include polydimethylsiloxanes (PDMSs) onto which are grafted, via a thiopropylene-type secondary bond, polyisobutyl (meth)acrylate polymer units.

Such polymers comprise polymers comprising at least one group of formula: 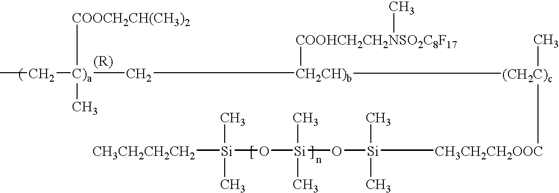
in which
a, b, and c, which may be identical or different, are each chosen from numbers ranging from 1 to 100,000; and the end groups, which may be identical or different, are each chosen from linear C₁-C₂₀ alkyl groups, C₃-C₂₀ branched-chain alkyl groups, C₃-C₂₀ aryl groups, linear C₁-C₂₀ alkoxy groups, and branched C₃-C₂₀ alkoxy groups.

Such polymers are disclosed, for example, in U.S. Pat. Nos. 4,972,037; 5,061,481; 5,209,924; 5,849,275; and 6,033,650 and PCT Patent Application Nos. WO 93/23446 and WO 95/06078.

Another family of silicone polymers having a polysiloxane skeleton grafted with non-silicone organic monomers, which may be suitable for use in accordance with certain embodiments, comprises silicone polymers comprising in their structure the unit of formula (V) below: 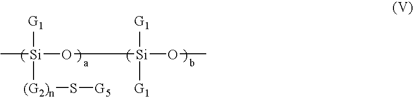
in which the radicals G₁ and G₂ have the same meaning as above; G₅ is a polymer residue resulting from the (homo)polymerization of at least one ethylenically unsaturated hydrophobic monomer or from the copolymerization of at least one ethylenically unsaturated anionic monomer and of at least one ethylenically unsaturated hydrophobic monomer; n is equal to 0 or 1; a is an integer ranging from 0 to 50; b is an integer ranging from 10 to 350; on condition that a is other than 0.

The unit of formula (V) above may have at least one, and for example all, of the following characteristics:
the radicals G₁ denote an alkyl radical, such as a methyl radical; and
n is not zero, and the radicals G₂ represent a C₁-C₃ divalent radical, such as a propylene radical.

The number-average molar mass of the silicone polymers with a polysiloxane skeleton grafted with non-silicone organic monomers disclosed herein may range from 10,000 to 1,000,000, such as from 10,000 to 100,000.

The composition may contain from 0.5% to 60% by weight, such as from 1% to 40% or from 2% to 30%, by weight of solids of film-forming polymer relative to the total weight of the composition.

More generally, the total amount of polymer should be an amount sufficient to form on the skin and/or the lips a cohesive film capable of following the movements of the skin and/or the lips without becoming detached or cracking.

When the polymer has a glass transition temperature that is too high for the desired use, a plasticizer may be combined therewith so as to lower this temperature of the mixture used. The plasticizer may be chosen from the plasticizers usually used in the are, for example from compounds that may be solvents for the polymer.

Physiologically Acceptable Medium

As used herein, the term “physiologically acceptable medium” denotes a non-toxic medium that may be applied to human skin and/or lips. The physiologically acceptable medium may be suited to the nature of the support onto which the composition is to be applied and also to the form in which the composition is intended to be packaged.

Aqueous Phase

The composition disclosed herein may comprise at least one aqueous medium, constituting an aqueous phase, which may form the continuous phase of the composition.

The aqueous phase may comprise water.

The aqueous phase may also comprise a mixture of water and of water-miscible organic solvent (miscibility in water to greater than 50% by weight at 25° C.), for instance lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones, and C₂-C₄ aldehydes.

The aqueous phase (water and optionally the water-miscible organic solvent) may be present in an amount ranging from 1% to 95% by weight, for example ranging from 3% to 80% by weight, or ranging from 5% to 60% by weight, relative to the total weight of the composition.

This aqueous phase may, where appropriate, be thickened, gelled, or structured by also incorporating therein a conventional aqueous-gelling agent, for example an aqueous gelling agent of mineral origin, for instance clay, and aqueous gelling agent of organic origin, for instance an aqueous-gelling polymer.

Such a medium may also comprise at least one volatile oil as defined below.

Fatty Phase

The composition, such as when the composition is intended to be applied to the lips, may comprise a fatty phase and, for example, at least one fatty substance that is liquid at room temperature (25° C.) and at atmospheric pressure and/or a fatty substance that is solid at room temperature and at atmospheric pressure, such as at least one of waxes and gums. The fatty phase may also contain structuring and gelling agents of oils of organic nature and/or lipophilic organic solvents.

According to one embodiment disclosed herein, the cosmetic composition is free of paraffin, of petroleum jelly, and of lanolin. The reason for this is that lanolins may have the drawback of being heat-sensitive and ultraviolet-sensitive, and may have a tendency to become oxidizing over time, with a release of unpleasant odor, which may limit their use in cosmetic compositions. Furthermore, when lanolins are combined with oils commonly used in cosmetics, the compositions obtained may have tack problems, which may be more pronounced when the oil used has a high viscosity.

The fatty phase of the composition disclosed herein may comprise, as liquid fatty substance, at least one oil chosen from volatiles and non-volatile oils.

As used herein, the term “volatile oil” means any oil capable of evaporating on contact with the skin in less than one hour, at room temperature and atmospheric pressure. The at least one volatile oil may be chosen from volatile cosmetic oils, which are liquid at room temperature, having a non-zero vapor pressure, at room temperature and atmospheric pressure, ranging for example from 0.01 to 300 mmHg (1.33 Pa to 40,000 Pa), for example greater than 0.3 mmHg (30 Pa).

As used herein, the term “non-volatile oil” means an oil that remains on the skin at room temperature and atmospheric pressure for at least several hours and that may have a vapor pressure of less than 0.01 mmHg (1.33 Pa).

The at least one oil chosen from volatile oils and non-volatile oils may be a hydrocarbon-based oil, such as plant oils, animal oils and silicone oils. As used herein, the term “hydrocarbon-based oil” means an oil mainly containing hydrogen and carbon atoms and optionally containing at least one atom chosen from oxygen, nitrogen, sulfur, and phosphorus atoms.

The volatile hydrocarbon-based oils may be chosen from at least one of the following: hydrocarbon-based oils containing from 8 to 16 carbon atoms, for example branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names Isopar® and Permethyl®, and branched C₈-C₁₆ esters such as isohexyl neopentanoate. Other volatile hydrocarbon-based oils, for instance petroleum distillates, for example those sold under the name Shell Solt by the company Shell, may also be used.

Volatile oils that may also be used include volatile silicones, for instance volatile linear or cyclic silicone oils, for example those with a viscosity ≦8 centistokes (8×10⁻⁶ m²/s) and those containing from 2 to 7 silicon atoms, these silicones optionally comprising alkyl or alkoxy groups containing from 1 to 10 carbon atoms. As volatile silicone oils that may be used, mention may be made of at least one of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, and dodecamethylpentasiloxane.

The volatile oil may be present in the composition disclosed herein in an amount ranging from 0.1% to 98% by weight, such as from 1% to 65% by weight, or from 2% to 50% by weight, relative to the total weight of the composition.

The non-volatile oils may be chosen from non-volatile, optionally fluoro, hydrocarbon-based oils and non-volatile silicone oils.

Non-volatile hydrocarbon-based oils that may be mentioned include:
hydrocarbon-based oils of animal origin,
hydrocarbon-based oils of plant origin, such as triglycerides comprising fatty acid esters of glycerol, the fatty acids of which may have varied chain lengths from C₄ to C₂₄, these chains optionally being linear or branched, and saturated or unsaturated; these oils may, for example, be chosen from wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil, musk rose oil, shea butter, and caprylic/capric acid triglycerides, for instance those sold by the company Stárineries Dubois and those sold under the names Miglyol® 810, 812, and 818 by the company Dynamit Nobel,
synthetic ethers containing from 10 to 40 carbon atoms;
linear or branched hydrocarbons of mineral or synthetic origin, such as at least one of petroleum jelly, polydecenes, and hydrogenated polyisobutene such as parleam, and squalane,
synthetic esters, for instance oils of formula R₁COOR₂ in which R₁ represents a linear or branched fatty acid residue containing from 1 to 40 carbon atoms and R₂ represents a hydrocarbon-based chain, which may be branched, containing from 1 to 40 carbon atoms, with the proviso that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters,
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol, and 2-undecylpentadecanol, and higher fatty acids such as oleic acid, linoleic acid, linolenic acid, and mixtures thereof.

The non-volatile silicone oils that may be used in the composition disclosed herein may be chosen from non-volatile polydimethylsiloxanes (PDMSs), polydimethylsiloxanes comprising alkyl or alkoxy groups, which are pendent and/or at the end of a silicone chain, these groups each containing from 2 to 24 carbon atoms, phenylsilicones, for instance phenyl trimethicones, phenyl dimethicones, phenyl trimethylsiloxy diphenylsiloxanes, diphenyl dimethicones, diphenyl methyldiphenyl trisiloxanes, and 2-phenylethyl trimethylsiloxysilicates, and mixtures thereof.

The non-volatile oils may be present in the composition disclosed herein in an amount ranging from 0.01% to 90% by weight, such as from 0.1% to 85% by weight or from 1% to 70% by weight, relative to the total weight of the composition.

The oils may be present in an amount ranging from 0.01% to 99% of the total weight of the composition, such as from 0.05% to 60% or from 1% to 35%.

In accordance with the present disclosure, oils may be included, which are other than the diol dimer ester and the acid ester described above, the molar weight of which is ranges from 650 to 10,000 g/mol, such as from 750 to 7,500 g/mol.

According to one embodiment, the composition disclosed herein comprises an oily phase comprising at least 5% by weight of an oil with a high molar mass, such as a molar mass ranging from 650 to 10,000 g/mol, such as from 750 to 7,500 g/mol.

The oil of high molar mass may be chosen from at least one of the following lipophilic polymers:
linear fatty acid esters with a total carbon number ranging from 35 to 70,
hydroxylated esters,
aromatic esters,
esters of a fatty alcohol and esters of fatty acids which are branched and of C₂₄-C₂₈,
silicone oils, and oils of plant origin.

The oil of high molar mass may be chosen from at least one of polybutylenes, hydrogenated polyisobutylenes, polydecenes, hydrogenated polydecenes, vinylpyrrolidone copolymers, such as the PVP/hexadecene copolymer, pentaerythrityl tetrapelargonate, polyglyceryl-2 triisostearate, tridecyl trimellitate, triisoarachidyl citrate, pentaerythrityl tetraisononanoate, glyceryl triisostearate, pentaerythrityl tetraisostearate, glyceryl tris(2-decyl)tetradecanoate, phenylsilicones, and sesame oil.

More generally, the fatty substance that is liquid at room temperature and at atmospheric pressure may be present in the composition in an amount ranging from 0.01% to 90% by weight, such as from 0.1% to 85% by weight, relative to the weight of the fatty phase.

The cosmetic composition may comprise, for example when it is intended to be applied to the lips, an oily phase with a refractive index of between 1.47 and 1.51, which may allow a relatively high gloss to be obtained.

As regards the fatty substance that is solid at room temperature and at atmospheric pressure, it may be chosen from at least one of waxes and gums. This solid fatty substance may be present in the composition in an amount ranging from 0.01% to 50%, such as from 0.1% to 40% or from 0.2% to 30%, by weight relative to the total weight of the fatty phase.

In certain embodiments, the composition may contain at least one wax.

As used herein, the term “wax” means a lipophilic fatty compound that is solid at room temperature (25° C.), which undergoes a reversible solid/liquid change of state, which has a melting point of greater than 30° C. which may be up to 200° C. and a hardness of greater than 0.5 MPa, and which has an anisotropic crystal organization in the solid state. By bringing the wax to its melting point, it may be possible to make it miscible with oils and to form a microscopically homogeneous mixture, but on returning the temperature of the mixture to room temperature, recrystallization of the wax in the oils of the mixture may be obtained.

The waxes that may be used in accordance with certain embodiments disclosed herein are compounds that are solid at room temperature, intended to structure the composition, for example in the form of a stick. The at least one wax may be chosen from hydrocarbon-based waxes, fluoro waxes, and silicone waxes and may be of plant, mineral, animal, and/or synthetic origin. For example, the at least one wax may have a melting point of greater than 40° C., such as greater than 45° C.

As waxes that may be used, mention may be made of those generally used in cosmetics: they may, for example, be chosen from at least one of natural origin, for instance beeswax, carnauba wax, candelilla wax, ouricurry wax, Japan wax, cork fibre wax, sugarcane wax, rice wax, montan wax, paraffin, lignite wax, microcrystalline wax, ceresin, ozokerite, hydrogenated oils, for instance jojoba oil, synthetic waxes, for instance the polyethylene waxes derived from the polymerization or copolymerization of ethylene and Fischer-Tropsch waxes, and fatty acid esters, for instance octacosanyl stearate, glycerides that are solid at 40° C., for example at 45° C., silicone waxes, for instance alkyl- or alkoxydimethicones containing an alkyl or alkoxy chain of 10 to 45 carbon atoms, poly(di)methylsiloxane esters that are solid at 40° C. and whose ester chain contains at least 10 carbon atoms, and mixtures thereof.

The composition disclosed herein may comprise at least one gum. The at least one gum that may be used may be in dissolved form in an oil, the polymers are solid at room temperature and the resins may be liquid or solid at room temperature.

As used herein, the term “gum” means a fatty substance that is in the form of a solid polymer at room temperature, with a weight-average molecular weight ranging from 50,000 to 1,000,000. The gum may be sold as a dispersion in an organic solvent such as silicone oil.

The nature and amount of the at least one gum or at least one wax depends on the desired mechanical properties and textures. As a guide, the at least one wax may be present in an amount ranging from 0.01% to 50%, such as from 2% to 40% or from 5% to 30%, by weight relative to the total weight of the composition.

The composition disclosed herein may further comprise at least one filler. As used herein, the term “filler” is intended to denote any organic and/or mineral compound introduced into the cosmetic composition in order to adjust its texture properties or, in other words, to control its rheological properties. Pigments and nacres, for example, are excluded from this definition.

According to one embodiment disclosed herein, the cosmetic compositions comprise less than 15% by weight, such as less than 10% by weight or less than 7% by weight, of at least one filler relative to the total weight of the composition.

The at least one filler may be chosen from spherical fillers, for instance talc, zinc stearate, mica, kaolin, polyamide (Nylon®) (Orgasol® from Atochem) powders, polyethylene powders, tetrafluoroethylene polymer (Teflon®) powders, starch, boron nitride, polymer microspheres such as those of polyvinylidene chloride/acrylonitrile, for instance Expancel® (Nobel Industrie), acrylic acid copolymers (Polytrap® from the company Dow Corning), silicone resin microbeads (for example Tospearls® from Toshiba), and organopolysiloxane elastomers.

The composition disclosed herein may also comprise at least one emulsifying surfactant present, for example, in an amount ranging from 0.1% to 30% by weight, such as from 5% to 15% by weight, relative to the total weight of the composition.

This at least one surfactant may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, for example pp. 347-377 of this reference, for the anionic and nonionic surfactants.

Surfactants that may be mentioned are chosen from:
nonionic surfactants chosen from fatty acids, fatty alcohols, polyethoxylated or polyglycerolated fatty alcohols such as polyethoxylated stearyl and cetylstearyl alcohol, fatty acid esters of sucrose, and alkylglucose esters, for example polyoxyethylenated C₁-C₆ alkyl glucose fatty esters, and mixtures thereof,
anionic surfactants chosen from C₁₆-C₃₀ fatty acids neutralized with amines, aqueous ammonia, or alkaline salts, and mixtures thereof.

Surfactants that allow oil-in-water or wax-in-water emulsions to be obtained may be used.

The composition disclosed herein may comprise at least one coloring agent, which may be present in an amount ranging from 0.01% to 40% by weight, such as from 0.01% to 30% by weight or from 0.05% to 25% by weight, relative to the total weight of the composition.

The at least one coloring agent may be chosen from at least one of pigments, water-soluble dyes, water-soluble nacres, liposoluble dyes, and liposoluble nacres.

As used herein, the term “pigments” should be understood as meaning white or colored, mineral or organic particles that are insoluble in the liquid hydrophilic phase, which are intended to color and/or opacify the composition. The term “nacres” should be understood as meaning iridescent particles produced for example by certain molluscs in their shell, or alternatively synthesized.

The pigments may be present in the composition in an amount ranging from 0.01% to 25% by weight, such as from 0.01% to 15% by weight or from 0.02% to 5% by weight, relative to the weight of the composition.

As mineral pigments that may be used, mention may be made of titanium oxide, zirconium oxide, cerium oxide, zinc oxide, iron oxide, chromium oxide, ferric blue, manganese violet, ultramarine blue, and chromium hydrate. Among the organic pigments that may be used, mention may be made of carbon black, D & C pigments, lakes based on cochineal carmine, lakes based on at least one of barium, strontium, calcium, and aluminium, and the diketone pyrrolopyrroles (DPP) described, for example, in the patent documents EP A 542 669, EP A 787 730, EP A 787 731, and WO A 96/08537. The amount and/or choice of the at least one pigments may be adjusted by taking into account the amount of nanotubes present in the cosmetic composition under consideration.

The nacres may be present in the composition in an amount ranging from 0.01% to 25% by weight, such as from 0.01% to 15% by weight or from 0.02% to 5% by weight, relative to the total weight of the composition.

The nacreous pigments may be chosen from white nacreous pigments such as mica coated with titanium or with bismuth oxychloride, colored nacreous pigments such as titanium mica with iron oxides, titanium mica for example with ferric blue or with chromium oxide, titanium mica with an organic pigment of the abovementioned type and nacreous pigments based on bismuth oxychloride.

The composition may also comprise at least one water-soluble or liposoluble dye in an amount ranging from 0.01% to 6% by weight, such as ranging from 0.01% to 3% by weight, relative to the total weight of the composition. The at least one liposoluble dye may be, for example, chosen from Sudan Red, DC Red 17, DC Green 6, β-carotene, soybean oil, Sudan Brown, DC Yellow 11, DC Violet 2, DC Orange 5, and quinoline yellow. The at least one water-soluble dye may be, for example, beetroot juice and methylene blue.

The composition disclosed herein may also comprise any ingredient conventionally used in the fields under consideration, such as in cosmetics and dermatology. These ingredients may be chosen from at least one of vitamins, antioxidants, thickeners, trace elements, softeners, sequestering agents, fragrances, basifying agents, acidifying agents, preserving agents, UV-screening agents, hydrophilic active agents, and lipophilic active agents. The amounts of these various ingredients may be those conventionally used in the fields under consideration, for example from 0.01% to 20% by weight relative to the total weight of the composition.

Needless to say, a person skilled in the art will take care to select this or these additional optional compound(s), and/or the amount thereof, such that the advantageous properties of the composition disclosed herein are not, or are not substantially, adversely affected by the envisaged addition.

The composition disclosed herein may be obtained according to the preparation processes conventionally used in cosmetics or dermatology.

The composition disclosed herein may be in the form of a solid composition, compacted or cast in stick or dish form, or a pasty or liquid composition. It may, for example, be in solid form, i.e., in hard form (which does not flow under its own weight) which has been cast or compacted, for example as a stick or a dish.

In the present case, it may be in the form of lipsticks, lip balms, cast foundations, concealer products, complexion “correctors” and “embellishers”, eyeshadows, and makeup rouges.

However, it may be in the form of a paste, a solid, or a cream. It may be an oil-in-water or water-in-oil emulsion, a solid or supple anhydrous gel, or in the form of a free or compacted powder, and may even be in two-phase form. According to one embodiment, the composition is in the form of an emulsion.

The composition disclosed herein may be in the form of a colored or uncolored composition, in the form of an antisun composition, makeup-removing composition, and/or a hygiene composition. The composition disclosed herein may comprise cosmetic active agents. It may then be used as a care and/or treatment base for the skin, for instance the hands and/or the face, for the lips (lip balms, for protecting the lips against the cold and/or sunlight and/or the wind), and/or as a deodorant. As cosmetic active agents that may be used, mention may be made of at least one agent chosen from vitamins A, E, C, and B3; provitamins, for instance D-panthenol; calmative active agents, for instance α-bisabolol, aloe vera, and allantoin; plant extracts; essential oils; protective or restructuring agents, for instance ceramides; refreshing active agents, for instance menthol and derivatives thereof; emollients (cocoa butter, dimethicone); moisturizers (arginine PCA); anti-wrinkle active agents; and essential fatty acids.

The composition disclosed herein may also be in the form of a makeup product for the skin, such as for facial skin, for instance a foundation, a blusher, a makeup such as a semi-permanent tattoo product, and/or a lip makeup product, for instance a lipstick or lip gloss, optionally having care and/or treatment properties; a makeup product for the integuments, for instance a nail varnish, a mascara or an eyeliner; and hair-coloring and/or haircare products.

Needless to say, the composition disclosed herein should be cosmetically acceptable, i.e., non-toxic and able to be applied to human skin, integuments and/or lips.

The examples of compositions below are given for illustrative purposes and without limiting nature.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

The following examples are intended to illustrate the invention without limiting the scope as a result.

### EXAMPLE 1

### Synthesis of a Polymer Particle Dispersion

A dispersion of non-crosslinked copolymer of methyl acrylate and of acrylic acid in an 85/15 ratio, in heptane, was prepared according to the method of Example 1 of European Patent No. EP A 749 746. When the polymerization was complete, hydrogenated polyisobutene was added, and the heptane was distilled off under vacuum.

A dispersion of poly(methyl acrylate/acrylic acid) particles surface-stabilized in hydrogenated polyisobutene with a polystyrene/copoly(ethylene-propylene) diblock copolymer sold under the name Kraton® G1701, having a solids content of 21% by weight and a mean particle size equal to 150 nm, was thus obtained.

### EXAMPLE 2

### Lipstick

| Chemical name | Example 2 |
|---|---|
| Dispersion of polymer of Example 1 | 30 |
| 2-Decyltetradecanoic acid triglyceride | 2.02 |
| Dilinoleyl diol dimer/dilinoleic | 10 |
| dimer copolymer | |
| Octyldodecanol | 9 |
| BHT | 0.07 |
| Mixture of parabens | 0.4 |
| Polycaprolactone of MW 1,250 g/mol | 9 |
| (Capa ® 1215 from Solvay) | |
| Polystearyl acrylate (Intelimer ® IPA | — |
| 13-1 from Landec) | |
| Vinylpyrrolidone/eicosene copolymer | 6 |
| Microcrystalline wax | 10 |
| Polyethylene wax | 2 |
| Polymethylene wax of m.p. 40° C. | 10 |
| Pigments | 6.03 |
| Dimethicone-coated silica | 5 |
| Fragrance | 0.48 |
| TOTAL | 100 |

Procedure:

All the starting materials were weighed out into an oil-circulated jacketed heating vessel and were then heated with stirring (turbomixer).

After total melting of the materials and homogenization of the mixture, it was ground five times in succession using a three-roll mill. The paste obtained was left to stabilize for 24 hours at 20° C. and was then packaged in heating bags.

Cosmetic Evaluation (In Vivo):

The formula was tested in a half-lip test on seven women. The testers graded on a scale from 1 to 10 the level of migration of the formula after one hour (1=little migration, 10=substantial migration). The migration of Example 2 was equal to 1.14.

Evaluation (In Vitro):

The formula was tested in vitro according to the “Push & Pull” test, which comprises evaluating the resistance of the formula to water and to oil. The results were as follows:

| | Example 2 |
|---|---|
| Resistance to pressure | 102.25 |
| Resistance to pressure + wiping | 60.43 |

### EXAMPLE 3

### Lipstick

| | |
|---|---|
| Hydrogenated isoparaffin (Parleam from NOF) | 5.7 |
| Dilinoleyl diol dimer/dilinoleic dimer copolymer | 5.7 |
| (Lusplan DD-DA 5 from NFC) | |
| Mixture of hydrogenated plant oils | 20 |
| (soybean/coconut/palm/rapeseed) | |
| Lipex ® 451 from Karlshamns\ | 10.5 |
| Polyethylene wax (MW 500) | |
| Ozokerite wax (Ozokerite Wax SP 1020 P from Strahl & Pitsch) | 2.8 |
| Pigments | 10.2 |
| Polybutene (Indopol ® H 1500) | 5.0 |
| Dispersion of polymer in isohexadecane | 40 |

Preparation of the Polymer Dispersion:

A dispersion of non-crosslinked copolymer of methyl acrylate and of acrylic acid in a 95/5 ratio in isododecane was prepared according to the method of Example 1 of European Patent No. EP A 749 746, replacing the heptane with isododecane. A dispersion of poly(methyl acrylate/acrylic acid) particles surface-stabilized in isododecane with a polystyrene/copoly(ethylene-propylene) diblock copolymer sold under the name Kraton® G1701, having a solids content of 25% by weight, was thus obtained.

The lipstick had good staying power, a good level of gloss, and did not transfer.

1. A cosmetic composition comprising, in a physiologically acceptable medium:
at least one ester of a diol dimer and of at least one acid chosen from C₄ to C₃₄ monocarboxylic acids and C₄ to C₃₄ dicarboxylic acids, and at least one film-forming polymer.

2. The composition according to claim 1, wherein the monocarboxylic acids are chosen from:
saturated linear acids,
fatty acids,
hydroxy acids,
cyclic acids,
and mixtures thereof.

3. The composition according to claim 2, wherein the saturated linear acids are chosen from butanoic acid, pentanoic acid, hexanoic acid, heptanoic acid, octanoic acid, nonanoic acid, decanoic acid, undecanoic acid, dodecanoic acid, tridecanoic acid, tetradecanoic acid, heptadecanoic acid, hexadecanoic acid, pentadecanoic acid, octadecanoic acid, nonadecanoic acid, eicosanoic acid, docosanoic acid, and tetracosanoic acid.

4. The composition according to claim 2, wherein the hydroxy acids are chosen from 2-hydroxybutanoic acid, 2-hydropentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxytetradecanoic acid, 2-hydroxyhexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid, and 2-hydroxytetracosanoic acid.

5. The composition according to claim 2, wherein the cyclic acids are chosen from cyclohexanoic acid, hydrogenated rosin, rosin, abietic acid, hydrogenated abietic acid, benzoic acid, p-oxybenzoic acid, p-aminobenzoic acid, cinnamic acid, p-methoxycinnamic acid, salicylic acid, gallic acid, pyrrolidonecarboxylic acid, and nicotinic acid.

6. The composition according to claim 2, wherein the fatty acids are chosen from:
branched fatty acids,
unsaturated linear C₈ to C₃₄ fatty acids, and
fatty acids of natural origin.

7. The composition according to claim 6, wherein the branched fatty acids are chosen from isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyidodecanoic acid, 2-decyltetradecanoic acid, 2-dodecylhexadecanoic acid, 2-tetradecyloctadecanoic acid, and 2-hexadecyloctadecanoic acid.

8. The composition according to claim 6, wherein the unsaturated linear C₈ to C₃₄ fatty acids are chosen from undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, and arachidonic acid.

9. The composition according to claim 6, wherein the fatty acids of natural origin are chosen from the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil, and of mink oil.

10. The composition according to claim 1, wherein the dicarboxylic acids are chosen from:
compounds of formula (I) HOOC—(CH₂)ₙ—COOH in which n is an integer ranging from 1 to 16, and
diacid dimers obtained by dimerization of at least one unsaturated monocarboxylic acid.

11. The composition according to claim 10, wherein n is an integer ranging from 3 to 16.

12. The composition according to claim 10, wherein the at least one unsaturated monocarboxylic acid is a C₈ to C₃₄ unsaturated fatty acid.

13. The composition according to claim 12, wherein the at least one unsaturated monocarboxylic acid is a C₁₈ unsaturated fatty acid.

14. The composition according to claim 12, wherein the unsaturated fatty acid is chosen from undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, and arachidonic acid, and mixtures thereof.

15. The composition according to claim 10, wherein the dicarboxylic acid is chosen from a diacid dimer obtained by dimerization of at least one unsaturated monocarboxylic acid.

16. The composition according to claim 15, wherein the diacid dimer is dilinoleic diacid.

17. The composition according to claim 10, wherein the diacid dimer is saturated.

18. The composition according to claim 1, wherein the diol dimer is derived from hydrogenation of a diacid dimer.

19. The composition according to claim 18, wherein the diacid dimer is derived from dimerization of an unsaturated fatty acid.

20. The composition according to claim 19, wherein the unsaturated fatty acid is a C₁₈ unsaturated fatty acid.

21. The composition according to claim 1, wherein the diol dimer is derived from hydrogenation of dilinoleic diacid.

22. The composition according to claim 18, wherein the diol dimer is saturated.

23. The composition according to claim 10, wherein the diacid dimer is identical to a diacid dimer from which the diol dimer is derived.

24. The composition according to claim 1, wherein the ester is a compound of formula (II):
HO—R¹—(—OCO—R²—COO—R¹—)ₕ—OH (II)
in which:
R¹ is a diol dimer residue obtained by hydrogenation of dilinoleic diacid,
R² is a hydrogenated dilinoleic diacid residue, and
h is an integer ranging from 1 to 9.

25. The composition according to claim 1, wherein the at least one ester is present in an amount ranging from 1% to 99% by weight, relative to the total weight of the composition.

26. The composition according to claim 25, wherein the at least one ester is present in an amount ranging from 10% to 35% by weight, relative to the total weight of the composition.

27. The composition according to claim 1, wherein the at least one film-forming polymer is chosen from liposoluble and amorphous homopolymers and copolymers of olefins, of cycloolefins, of butadiene, of isoprene, of styrene, of ethers, of vinyl esters, of vinyl amides, of (meth)acrylic acid esters, and of (meth)acrylic amides, containing a linear, branched, or cyclic C₄₋₅₀ alkyl group.

28. The composition according to claim 1, wherein the at least one film-forming polymer is chosen from homopolymers and copolymers obtained from monomers chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, and stearyl (meth)acrylate, and mixtures thereof.

29. The composition according to claim 1, wherein the at least one film-forming polymer is chosen from alkyl acrylate/cycloalkyl copolymers and vinylpyrrolidone/decadecene compolymers.

30. The composition according to claim 1, wherein the at least one film-forming polymer is chosen from amorphous and liposoluble polycondensates, optionally comprising at least one group donating hydrogen interactions.

31. The composition according to claim 30, wherein the at least one film-forming polymer is chosen from polyesters containing C₄₋₅₀ alkyl side chains, polyesters resulting from the condensation of fatty acid dimers, and polyesters comprising a silicone-based segment in the form of an end group, graft or block, which is solid at room temperature.

32. The composition according to claim 1, wherein the at least one film-forming polymer is chosen from amorphous and liposoluble polysaccharides comprising alkyl side chains chosen from ether and ester side chains and grafted silicone-acrylic polymers chosen from grafted-silicone polymers having a silicone skeleton and acrylic grafts and grafted silicone-polymers having an acrylic skeleton and silicone grafts.

33. The composition according to claim 32, wherein the alkyl side chain, is ethylcellulose.

34. The composition according to claim 1, wherein the at least one film-forming polymer comprises at least one fluoro group.

35. The composition according to claim 34, wherein the at least one film-forming polymer is chosen from alkyl (meth)acrylate/perfluoroalkyl (meth)acrylate copolymers.

36. The composition according to claim 1, wherein the at least one film-forming polymer is chosen from polymers and copolymers resulting from the polymerization or copolymerization of an ethylenic monomer comprising at least one ethylenic bond, which is optionally conjugated.

37. The composition according to claim 36, wherein the at least one film-forming polymer is chosen from polystyrene/copoly(ethylene/butylene)s.

38. The composition according to claim 1, wherein the at least one film-forming polymer is chosen from polymers with a non-silicone-based organic skeleton grafted with monomers containing a polysiloxane.

39. The composition according to claim 1, wherein the at least one film-forming polymer is chosen from silicone-based polymers grafted with non-silicone-based organic monomers.

40. The composition according to claim 1, wherein the at least one film-forming polymer is chosen from silicone-based polyamides.

41. The composition according to claim 1, wherein the at least one film-forming polymer is chosen from silicone resins.

42. The composition according to claim 41, wherein the at least one film-forming polymer is chosen from at least one of siloxysilicates and polysilsesquioxanes.

43. The composition according to claim 1, wherein the at least one film-forming polymer is a block polymer comprising at least one first block and at least one second block that have different glass transition temperatures (Tg), the first and second blocks being linked together via an intermediate block comprising at least one constituent monomer of the first block and at least one constituent monomer of the second block.

44. The composition according to claim 43, wherein the block polymer comprises at least one first block with a glass transition temperature (Tg) of greater than or equal to 40° C. and at least one second block with a glass transition temperature of less than or equal to 20° C.

45. The composition according to claim 44, wherein the first block is derived from at least one monomer chosen from methyl methacrylate, isobutyl methacrylate, and isobornyl (meth)acrylate.

46. The composition according to claim 44, wherein the second block is derived from at least one monomer chosen from alkyl acrylates in which the alkyl chain contains from 0.1 to 10 carbon atoms, with the proviso that the alkyl chain is not a butyl group.

47. The composition according to claim 44, wherein at least one of the first and second blocks comprises at least one unit derived from an additional monomer chosen from hydrophilic monomers and ethylenically unsaturated monomers comprising at least one silicon atom.

48. The composition according to claim 47, wherein each of the first and second blocks of the block polymer comprises at least one monomer chosen from (meth)acrylic acid esters and optionally at least one monomer chosen from (meth)acrylic acid.

49. The composition according to claim 43, wherein each of the first and second blocks of the block polymer is totally derived from at least one monomer chosen from acrylic acid, (meth)acrylic acid esters, and optionally (meth)acrylic acid.

50. The composition according to claim 1, further comprising a fatty phase comprising polymer particles that are solid and insoluble in the fatty phase at a temperature of 25° C.

51. The composition according to claim 50, wherein the polymer particles are not wax particles.

52. The composition according to claim 50, wherein the polymer particles have a mean size ranging from 5 to 800 nm.

53. The composition according to claim 50, wherein the polymer particles are hydrocarbon-based polymer particles.

54. The composition according to claim 50, wherein the polymer particles are insoluble in water-soluble alcohols.

55. The composition according to claim 50, wherein the polymer particles are chosen from polyurethanes, polyurethane-acrylics, polyureas, polyurea/polyurethanes, polyester-polyurethanes, polyether-polyurethanes, polyesters, polyester amides, fatty-chain polyesters, alkyds, acrylic polymers, acrylic copolymers, vinyl polymers, vinyl copolymers, acrylic-silicone copolymers, polyacrylamides, silicone polymers, and fluoro polymers, and mixtures thereof.

56. The composition according to claim 50, wherein the polymer particles, as solids, are present in the composition in an amount ranging from 5% to 40% by weight, relative to the total weight of the composition.

57. The composition according to claim 56, wherein the polymer particles, as solids, are present in the composition in an amount ranging from 8% to 30% by weight, relative to the total weight of the composition.

58. The composition according to claim 50, further comprising at least one stabilizer chosen from block polymers, grafted polymers, and random polymers.

59. The composition according to claim 58, wherein the at least one stabilizer is a diblock polymer.

60. The composition according to claim 50, wherein the fatty phase comprises less than 40% by weight of at least one volatile oil, relative to the total weight of the composition.

61. The composition according to claim 60, wherein the fatty phase comprises less than 10% by weight of at least one volatile oil, relative to the total weight of the composition.

62. The composition according to claim 50, wherein the fatty phase is free of volatile oil.

63. The composition according to claim 50, wherein the fatty phase comprises at least one non-volatile oil.

64. The composition according to claim 63, wherein the at least one non-volatile oil is a hydrocarbon-based oil.

65. The composition according to claim 63, wherein the at least one non-volatile oil is present in an amount ranging from 5% to 80% by weight relative to the total weight of the composition.

66. The composition according to claim 65, wherein the at least one non-volatile oil is present in an amount ranging from 15% to 30% by weight, relative to the total weight of the composition.

67. The composition according to claim 63, wherein the at least one non-volatile oil is apolar.

68. The composition according to claim 67, wherein the at least one non-volatile oil is chosen from linear or branched hydrocarbons.

69. The composition according to claim 68, wherein the linear or branched hydrocarbons are chosen from liquid paraffin, liquid petroleum jelly, liquid naphthalene, and hydrogenated polyisobutene, and mixtures thereof.

70. The composition according to claim 1, further comprising a liquid fatty phase in which the at least one film-forming polymer is a grafted ethylenic polymer comprising an ethylenic skeleton that is insoluble in the liquid fatty phase and side chains that are covalently bonded to the said skeleton and that are soluble in the liquid fatty phase.

71. The composition according to claim 70, wherein the grafted ethylenic polymer is a grafted acrylic polymer.

72. The composition according to claim 70, wherein the grafted ethylenic polymer is an acrylic polymer that may be obtained by free-radical polymerization in an organic polymerization medium:
of at least one acrylic monomer, and optionally of at least one additional non-acrylic vinyl monomer, to form the said insoluble skeleton; and
of at least one macromonomer comprising a polymerizable end group to form the side chains, the said macromonomer having a weight-average molar mass of greater than or equal to 200 and wherein the polymerized macromonomer is present in the composition in an amount ranging from 0.05% to 20% by weight, relative to the total weight of the polymer.

73. The composition according claim 72, wherein the at least one acrylic monomer is chosen from the following monomers and the salts thereof:
(i) the (meth)acrylates of formula: 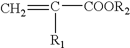
in which:
R₁ is chosen from hydrogen and methyl groups;
R₂ is a group chosen from:
linear or branched alkyl groups containing from 1 to 6 carbon atoms, optionally comprising in its chain at least one hetero atom chosen from O, N, and S; and/or optionally comprising at least one substituent chosen from —OH, halogen atoms, and —NR′R″ wherein R′ and R″, which may be identical or different, are chosen from linear or branched C₁-C₄ alkyls; and/or optionally substituted with at least one polyoxyalkylene group comprising a repetition of 5 to 30 oxyalkylene units;
cyclic alkyl groups containing from 3 to 6 carbon atoms, optionally comprising in its chain at least one hetero atom chosen from O, N, and S, and/or optionally comprising at least one substituent chosen from OH and halogen atoms;
(ii) the (meth)acrylamides of formula: 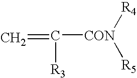
in which:
R₃ is chosen from hydrogen and methyl groups;
R₄ and R₅, which may be identical or different, are chosen from hydrogen and linear or branched alkyl groups containing from 1 to 6 carbon atoms, optionally comprising at least one substituent chosen from —OH, halogen atoms, and —NR′R″ wherein R′ and R″, which may be identical or different, are chosen from linear or branched C₁-C₄ alkyls; or
R₄ is a hydrogen atom and R₅ is a 1,1-dimethyl-3-oxobutyl group;
(iii) (meth)acrylic monomers comprising at least one functional group chosen from carboxylic acid, phosphoric acid, and sulfonic acid functional groups.

74. The composition according to claim 73, wherein the at least one acrylic monomer is chosen from methyl, ethyl, propyl, butyl, and isobutyl (meth)acrylates; methoxyethyl (meth)acrylates), ethoxyethyl (meth)acrylates; trifluoroethyl methacrylate; dimethylaminoethyl methacrylate; diethylaminoethyl methacrylate; 2-hydroxypropyl (meth)acrylate; 2-hydroxyethyl (meth)acrylate; dimethylaminopropylmethacrylamide; and the salts thereof.

75. The composition according to claim 73, wherein the at least one acrylic monomer is chosen from methyl acrylate, methoxyethyl acrylate, methyl methacrylate, 2-hydroxyethyl methacrylate, (meth)acrylic acid, and dimethylaminoethyl methacrylate, and mixtures thereof.

76. The composition according to claim 70, wherein the grafted ethylenic polymer comprises (meth)acrylic acid.

77. The composition according to claim 72, wherein the at least one acrylic monomer comprises at least (meth)acrylic acid and at least one monomer chosen from:
(i) the (meth)acrylates of formula: 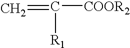
in which:
R₁ is chosen from hydrogen and methyl groups;
R₂ is a group chosen from:
linear or branched alkyl groups containing from 1 to 6 carbon atoms, optionally comprising in its chain at least one hetero atom chosen from O, N, and S; and/or optionally comprising at least one substituent chosen from —OH, halogen atoms, and —NR′R″ wherein R′ and R″, which may be identical or different, are chosen from linear or branched C₁-C₄ alkyls; and/or optionally substituted with at least one polyoxyalkylene group comprising a repetition of 5 to 30 oxyalkylene units;
cyclic alkyl groups containing from 3 to 6 carbon atoms, optionally comprising in its chain at least one hetero atom chosen from O, N, and S, and/or optionally comprising at least one substituent chosen from OH and halogen atoms; and
(ii) the (meth)acrylamides of formula: 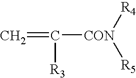
in which:
R₃ is chosen from hydrogen and methyl groups;
R₄ and R₅, which may be identical or different, are chosen from hydrogen and linear or branched alkyl groups containing from 1 to 6 carbon atoms, optionally comprising at least one substituent chosen from —OH, halogen atoms, and —NR′R″ wherein R′ and R″, which may be identical or different, are chosen from linear or branched C₁-C₄ alkyls; or
R₄ is a hydrogen atom and R₅ is a 1,1-dimethyl-3-oxobutyl group.

78. The composition according to claim 72, wherein the at least one acrylic monomer comprises at least (meth)acrylic acid and at least one monomer chosen from C₁-C₃ alkyl (meth)acrylates.

79. The composition according to claim 72, wherein the grafted acrylic polymer may be obtained by free-radical polymerization of at least one acrylic monomer and of at least one additional non-acrylic vinyl monomer and of the said macromonomer.

80. The composition according to claim 79, wherein the at least one additional non-acrylic vinyl monomer is chosen from:
vinyl esters of formula: R₆—COO—CH═CH₂
in which R₆ is chosen from linear or branched alkyl groups containing from 1 to 6 atoms and cyclic alkyl groups containing from 3 to 6 carbon atoms and/or optionally containing at least one aromatic group;
non-acrylic vinyl monomers comprising at least one of carboxylic acid, phosphoric acid, and sulfonic acid functional groups; and
non-acrylic vinyl monomers comprising at least one tertiary amine function,
and mixtures thereof.

81. The composition according to claim 72, wherein said at least one macromonomer comprises at one of the ends of its chain a polymerizable end group chosen from a vinyl group and a (meth)acrylate group.

82. The composition according to claim 70, wherein the liquid fatty phase comprises a liquid organic compound chosen from:
liquid organic compounds having a global solubility parameter according to the Hansen solubility space of less than or equal to 18 (MPa)^{1/2}; and
monoalcohols having a global solubility parameter according to the Hansen solubility space of less than or equal to 20 (MPa)^{1/2}, and
mixtures thereof.

83. The composition according to claim 1, wherein the at least one film-forming polymer is chosen from polyurethanes, polyurethane-acrylics, polyurethane-polyvinylpyrrolidones, polyester-polyurethanes, polyether-polyurethanes, polyureas, and polyurea/polyurethanes.

84. The composition according to claim 83, wherein the at least one film-forming polymer is chosen from aliphatic, cycloaliphatic, and aromatic polyurethane copolymers, copolymers of polyurea/polyurethane, and copolymers of polyurea comprising at least one block chosen from:
blocks of linear or branched, aliphatic, cycloaliphatic and/or aromatic polyester origin, blocks of aliphatic, cycloaliphatic, and/or aromatic polyether origin,
substituted or unsubstituted, branched or unbranched silicone-based blocks; and/or
blocks comprising at least one fluoro group.

85. The composition according to claim 83, wherein the at least one film-forming polymer is chosen from polyesters, polyesteramides, fatty-chain polyesters, polyamides, and epoxyester resins.

86. The composition according to claim 83, wherein the at least one film-forming polymer is chosen from acrylic polymers, acrylic copolymers, and vinyl polymers.

87. The composition according to claim 1, wherein the at least one film-forming polymer is chosen from:
proteins;
anionic, cationic, amphoteric, or nonionic chitin and chitosan polymers;
polymers of cellulose;
acrylic polymers and copolymers;
vinyl polymers;
copolymers of vinylpyrrolidone and of caprolactam;
polyvinyl alcohols; and
polymers of natural origin, optionally modified.

88. The composition according to claim 87, wherein the proteins are chosen from proteins of plant origin and proteins of animal origin.

89. The composition according to claim 87, wherein the polymers of cellulose are chosen from hydroxyethylcellulose, hydroxypropylcellulose, methylcellulose, ethylhydroxyethylcellulose and carboxymethylcellulose, and quaternized cellulose derivatives.

90. The composition according to claim 87, wherein the acrylic polymers and copolymers are chosen from polyacrylates and polymethacrylates.

91. The composition according to claim 87, wherein the vinyl polymers are chosen from polyvinylpyrrolidones, copolymers of methyl vinyl ether and of maleic anhydride, copolymers of vinyl acetate and of crotonic acid, and copolymers of vinylpyrrolidone and of vinyl acetate.

92. The composition according to claim 87, wherein the polymers of natural origin, optionally modified, are chosen from gum arabic, guar gum, xanthan derivatives, karaya gum, alginates, carrageenans, glycosaminoglycans, hyaluronic acid and derivatives thereof, shellac resin, sandarac gum, dammar resins, elemi gums, copal resins, deoxyribonucleic acid, mucopolysaccharides, and mixtures thereof.

93. The composition according to claim 1, further comprising at least one volatile oil.

94. The composition according to claim 93, wherein the at least one volatile oil is chosen from octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane, isododecane, isodecane, and isohexadecane.

95. The composition according to claim 93, wherein the at least one volatile oil is present in an amount ranging from 1% to 70% by weight, relative to the total weight of the composition.

96. The composition according to claim 95, wherein the at least one volatile oil is present in an amount ranging from 10% to 35% by weight, relative to the total weight of the composition.

97. The composition according to claim 1, further comprising at least one non-volatile oil.

98. The composition according to claim 97, wherein the at least one non-volatile oil is present in an amount ranging from 1% to 80% by weight, relative to the total weight of the composition.

99. The composition according to claim 98, wherein the at least one non-volatile oil is present in an amount ranging from 10% to 50% by weight, relative to the total weight of the composition.

100. The composition according to claim 1, wherein the at least one film-forming polymer is present in an amount ranging from 0.5% to 60% by weight of solids relative to the total weight of the composition.

101. The composition according to claim 100, wherein the at least one film-forming polymer is present in an amount ranging from 2% to 30% by weight of solids relative to the total weight of the composition.

102. The cosmetic composition according to claim 1, further comprising at least one dyestuff chosen from water-soluble dyes and pulverulent dyestuffs.

103. The composition according to claim 102, wherein the at least one dyestuff is present in an amount ranging from 0.01% to 50% by weight relative to the weight of the composition.

104. The composition according to claim 103, wherein the at least one dyestuff is present in an amount ranging from 0.01% to 30% by weight relative to the weight of the composition.

105. The composition according to claim 1, further comprising at least one fatty substance chosen from waxes, pasty fatty substances, and gums.

106. The composition according to claim 1, further comprising at least one cosmetic ingredient chosen from vitamins, thickeners, gelling agents, trace elements, softeners, sequestering agents, fragrances, acidifying agents, basifying agents, preserving agents, sunscreens, surfactants, antioxidants, fibers, agents for preventing hair loss, eyelash care agents, antidandruff agents, and propellants.

107. The cosmetic composition according to claim 1, wherein the composition is a form chosen from suspensions, dispersions, solutions, gels, emulsions, creams, pastes, mousses, vesicular dispersions, two-phase lotions, multi-phase lotions, sprays, powders, pastes, sticks, and cast solids.

108. The composition according to claim 1, wherein the composition is in anhydrous form.

109. The composition according to claim 1, wherein the composition is a makeup and/or care composition for keratin materials.

110. The composition according to claim 109, wherein the makeup and/or care composition is chosen from an eye makeup product, a mascara, a lip makeup product, a complexion makeup product, and a nail makeup product.

111. A process for making up and/or caring for the skin, the lips, and/or the integuments comprising
applying to the skin, the lips and/or the integuments at least one cosmetic composition comprising, in a physiologically acceptable medium,
at least one ester of a diol dimer and of at least one acid chosen from C₄ to C₃₄ monocarboxylic acids and C₄ to C₃₄ dicarboxylic acids, and
at least one film-forming polymer.
